(19) United States
(12) Patent Application Publication
(10) Pub. No.: US 2023/0395262 A1
(43) Pub. Date: Dec. 7, 2023

(54) SYSTEMS AND METHODS FOR MONITORING IMPLANTABLE MEDICAL DEVICE USAGE AND TREATMENT PLAN ADHERENCE

(71) Applicant: INSPIRE MEDICAL SYSTEMS, INC., Golden Valley, MN (US)

(72) Inventors: Kent Lee, Minneapolis, MN (US); Matheus Araujo, Golden Valley, MN (US); John Rondoni, Plymouth, MN (US)

(73) Assignee: INSPIRE MEDICAL SYSTEMS, INC., Golden Valley, MN (US)

(21) Appl. No.: 18/205,744

(22) Filed: Jun. 5, 2023

Related U.S. Application Data
(60) Provisional application No. 63/348,952, filed on Jun. 3, 2022.

(51) Int. Cl. G16H 50/30; G16H 10/60; G16H 50/70; G16H 20/40; A61B 5/00

(52) U.S. Cl. CPC G16H 50/30; G16H 10/60; A61B 5/4818; G16H 20/40; G16H 50/70

(57) ABSTRACT

Systems and methods for monitoring implantable medical device usage and treatment plan adherence.

### CROSS-REFERENCE TO RELATED APPLICATIONS

This application claims the benefit of the filing date of U.S. Provisional Application Ser. No. 63/348,952, filed Jun. 3, 2022 and entitled “Systems And Methods For Monitoring Implantable Medical Device Usage And Treatment Plan Adherence,” the entire teachings of which are incorporated herein by reference.

### BACKGROUND

Some implantable medical devices may communicate with external devices to provide information regarding operation of the implantable medical device within the patient, status of the patient, and the like.

### BRIEF DESCRIPTION OF THE DRAWINGS

FIG. 1 is a block diagram schematically representing an example arrangement, deployable in an example method of (or as an example system for) patient management to facilitate patient care.

FIG. 2 is a block diagram schematically representing an example adherence prediction engine.

FIG. 3 presents simplified representations of example adherence predictions.

FIG. 4 is a block diagram representing one example of a method for performing adherence prediction.

FIG. 5 is a block diagram schematically representing an example causation prediction engine.

FIG. 6A is a plot of daily therapy session durations for an example patient.

FIG. 6B is a plot of therapy session stimulation intensities for the example patient of FIG. 6A.

FIG. 6C is a plot of determined correlation coefficients for the example patient of FIGS. 6A and 6B.

FIG. 7 illustrates a report of therapy intensity discomfort zones for the example patient of FIGS. 6A-6C.

FIGS. 8 and 9 are example therapy intensity discomfort zone reports.

FIG. 10A is a block diagram schematically representing available actions of an example recommendations engine.

FIG. 10B is a block diagram schematically representing example sleep improvement actions for the recommendations engine of FIG. 10A.

FIG. 100 is a block diagram schematically representing example insomnia relief actions for the recommendations engine of FIG. 10A.

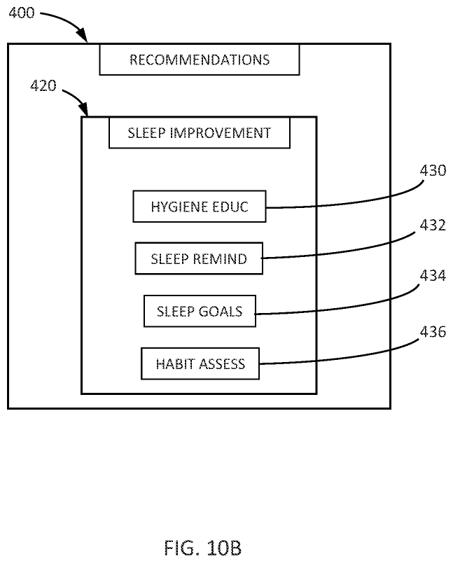

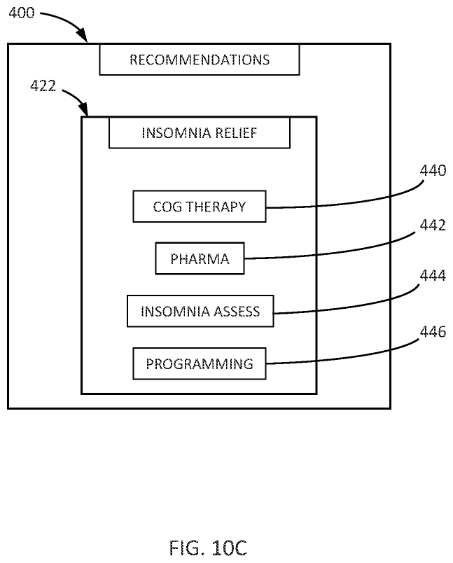

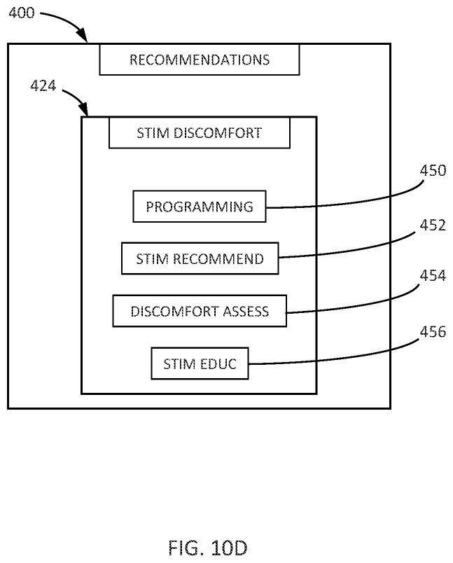

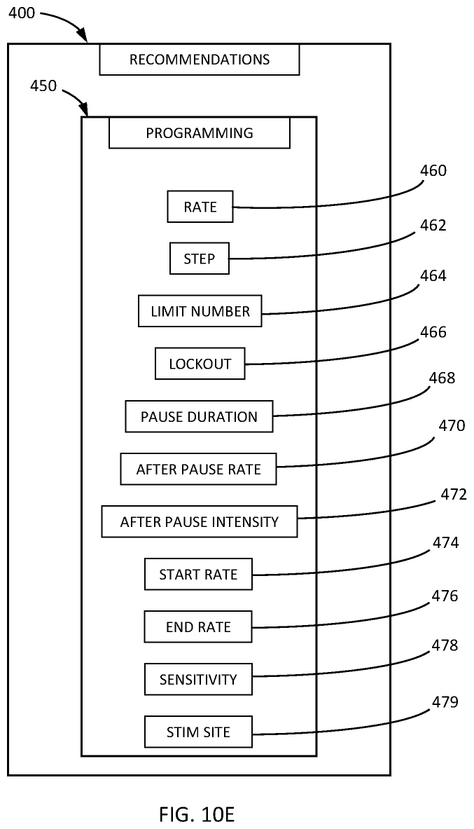

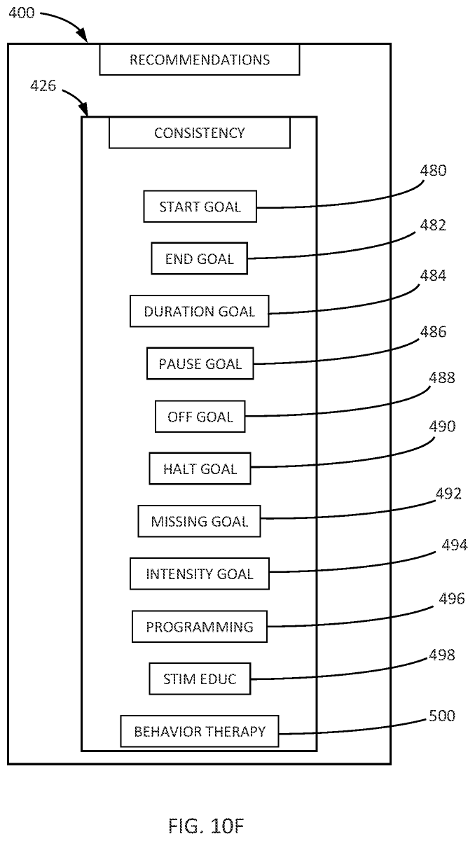

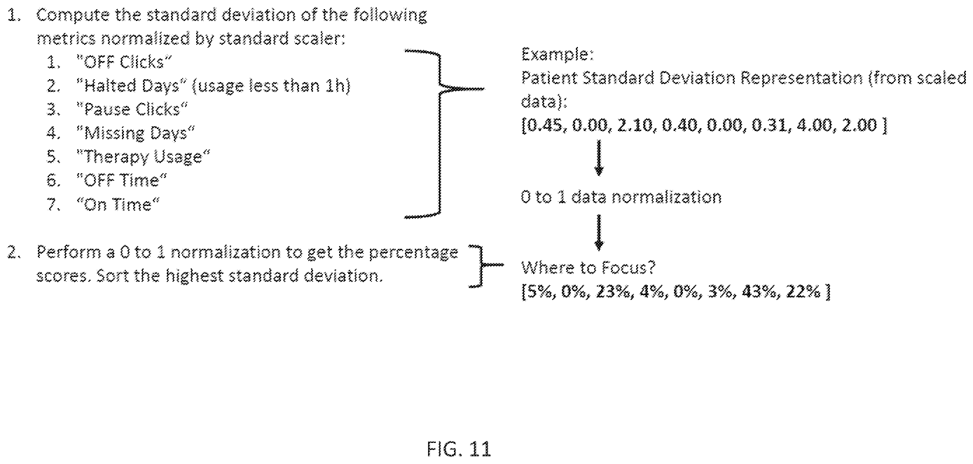

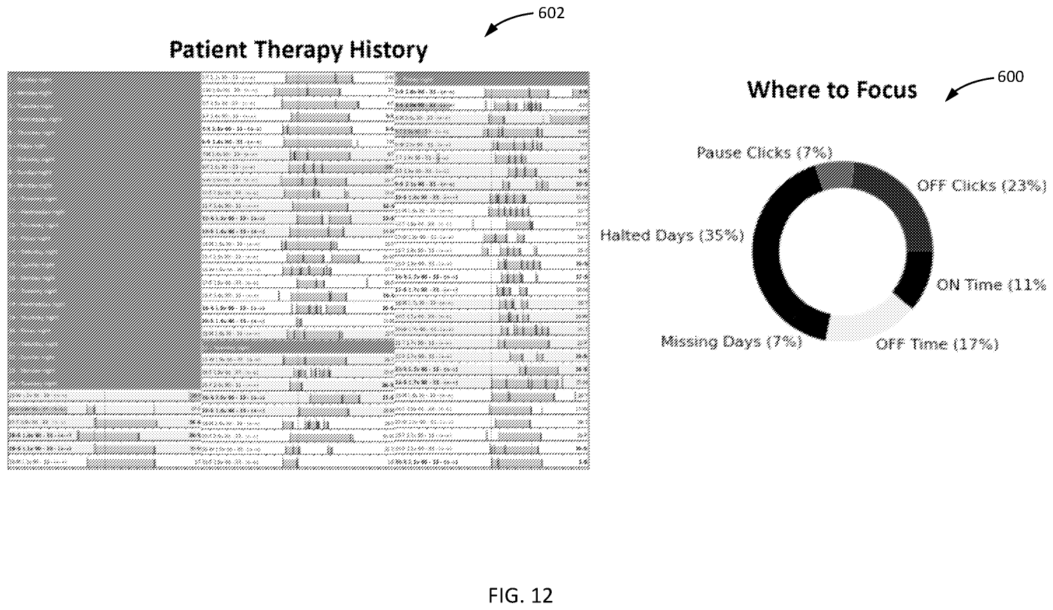

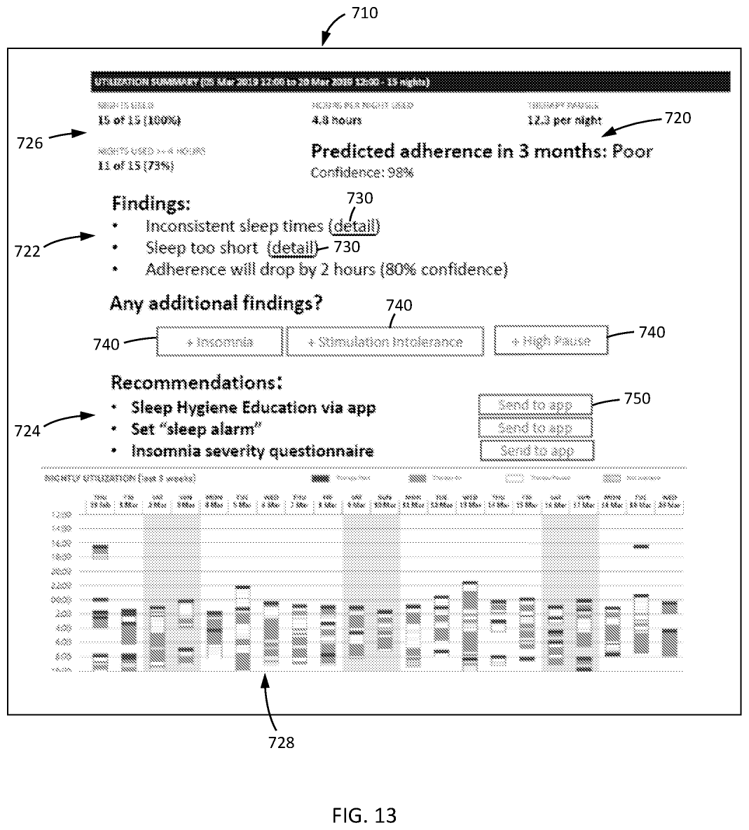

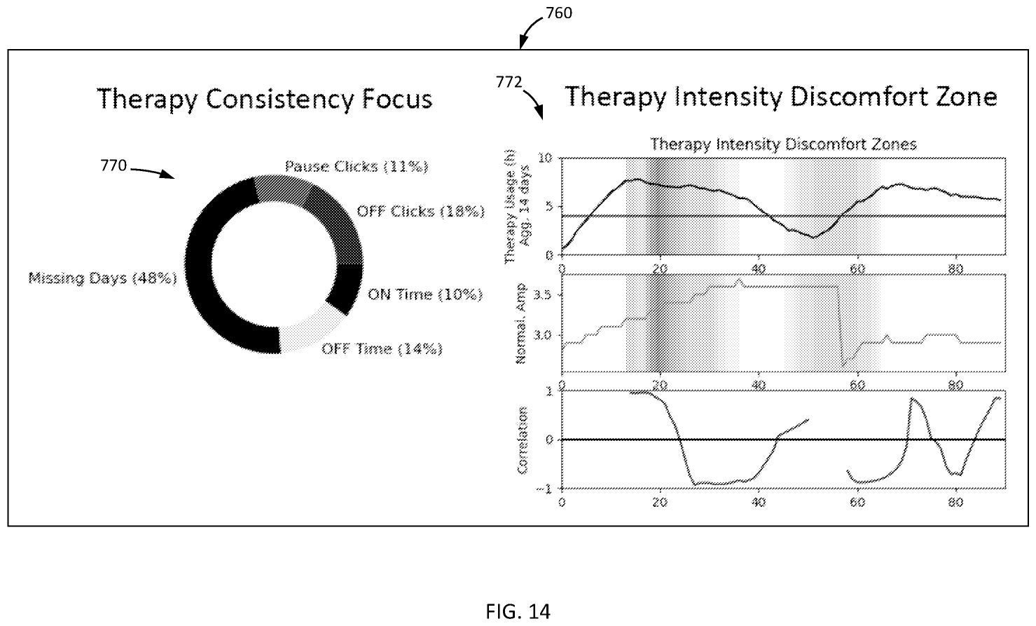

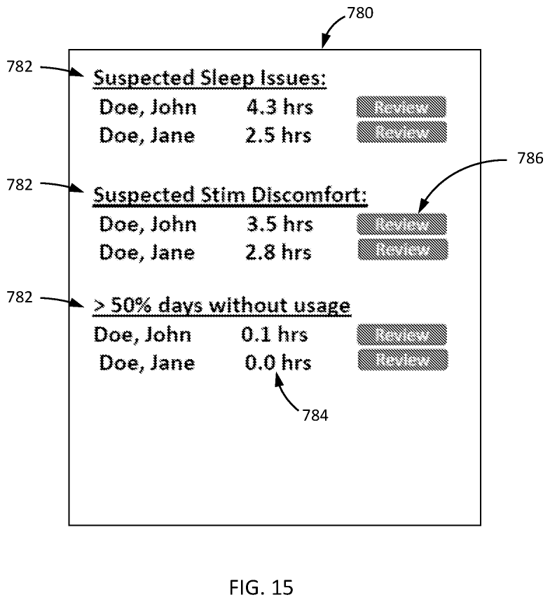

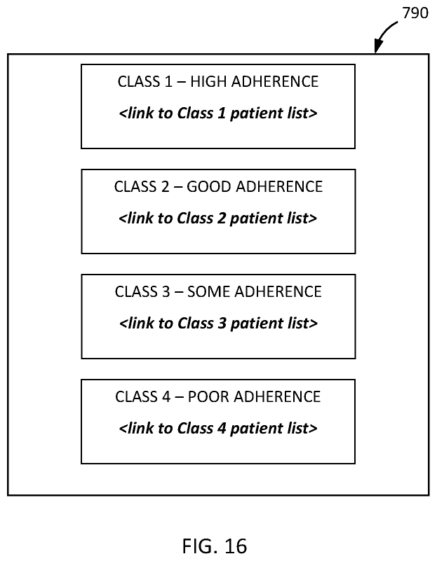

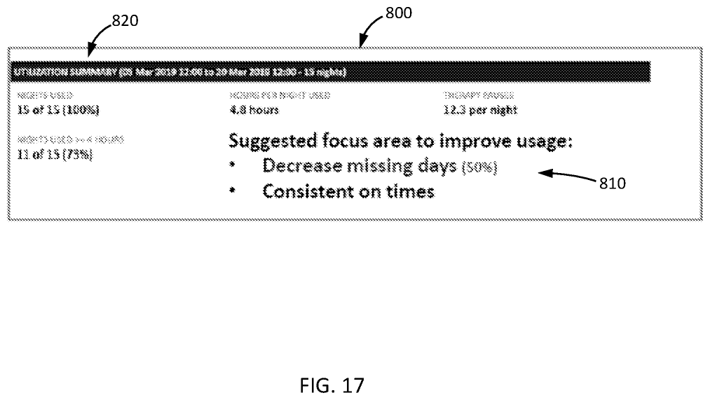

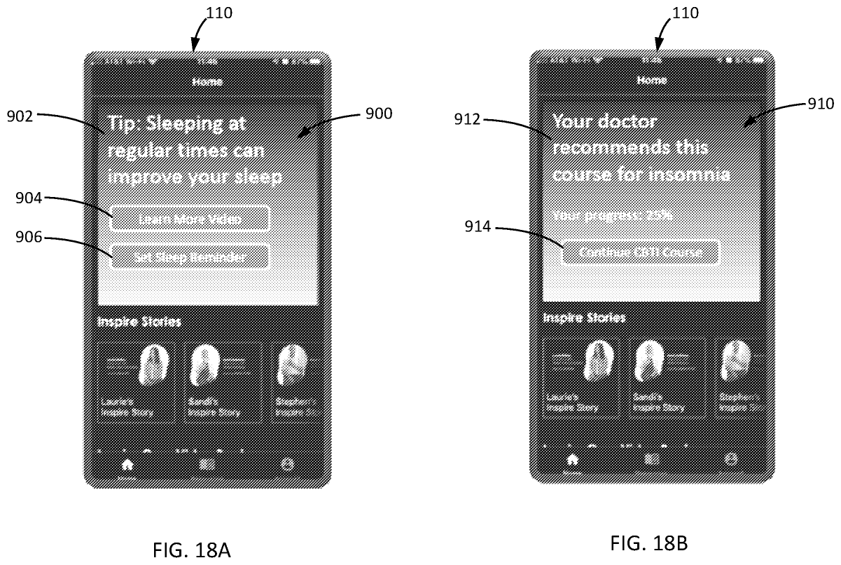

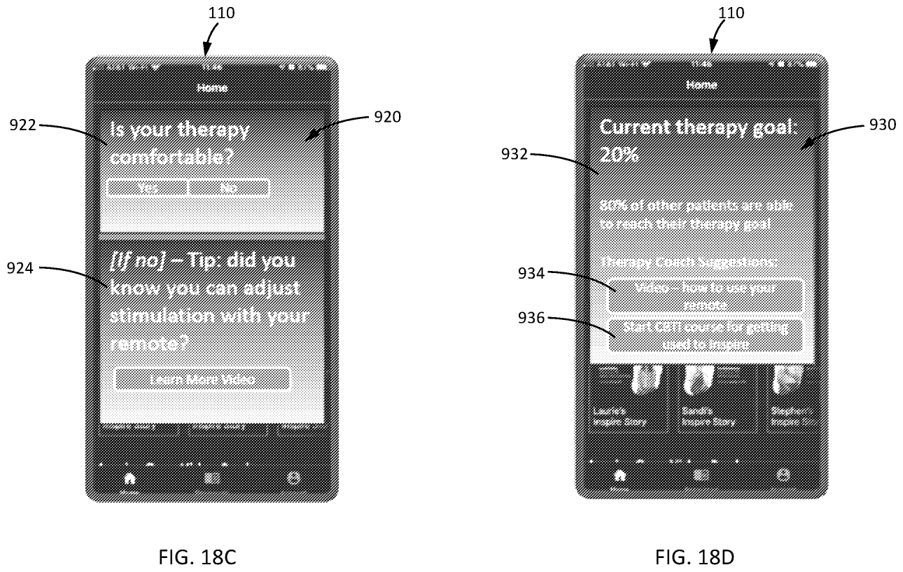

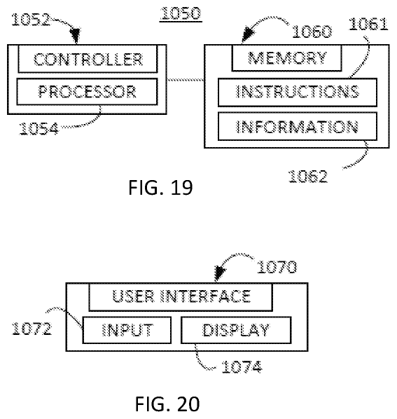

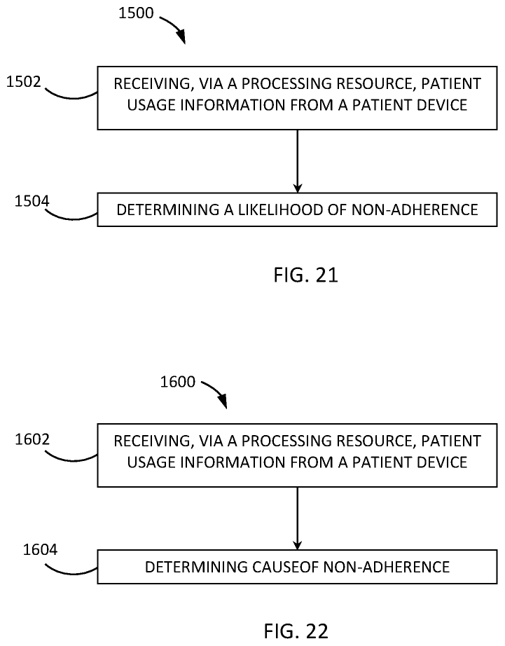

FIG. 10D is a block diagram schematically representing example stimulation discomfort actions for the recommendations engine of FIG. 10A.

FIG. 10E is a block diagram schematically representing example programming actions for the recommendations engine of FIG. 10A.

FIG. 10F is a block diagram schematically representing example consistency actions for the recommendations engine of FIG. 10A.

FIG. 11 schematically illustrates an example method of determining focused actions.

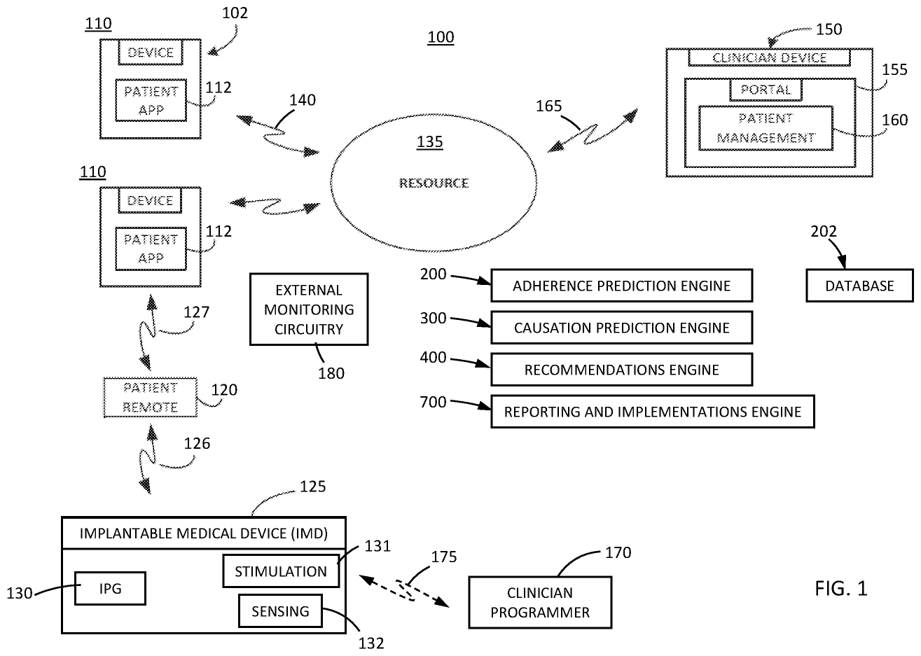

FIG. 12 illustrates an example report generated by the recommendations engine of FIG. 1.

FIG. 13 is a diagram schematically representing an example clinician report, for example as a graphical user interface (GUI).

FIG. 14 is a diagram schematically representing a further example clinician report, for example as a GUI.

FIG. 15 is a diagram schematically representing a further example clinician report, for example as a GUI.

FIG. 16 is a diagram schematically representing a further example clinician report, for example as a GUI.

FIG. 17 is a diagram schematically representing an example patient report, for example as a GUI.

FIGS. 18A-18D illustrate example patient user interface displays.

FIG. 19 is a block diagram schematically representing an example control portion.

FIG. 20 is a block diagram schematically representing an example user interface.

FIG. 21 is a flow diagram illustrating one example of a method for determining a likelihood of non-adherence.

FIG. 22 is a flow diagram illustrating one example of a method for determining a cause of non-adherence.

FIG. 23 is a flow diagram illustrating one example of a method for determining recommended action(s).

FIG. 24 is a diagram schematically representing patient anatomy and an example device and/or example method for stimulating an infrahyoid muscle (IHM)-innervating nerve and/or hypoglossal nerve.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to integrating information from different sources and/or pathways, etc. to enhance patient care in treating sleep disordered breathing (SDB) with stimulation energy delivered by an implantable medical device, for example to predict likelihood of patient adherence to an SDB treatment. In some such examples, the different sources and/or pathways may comprise patient management information, stimulation therapy information, comparative information, and/or third party diagnostic/monitoring information. It will be understood that at least some examples of the present disclosure use the predicted likelihood of patient adherence as feedback to take actions, such as delivering stimulation therapy, performing patient management actions, adjusting monitoring, and the like. Moreover, each action in turn, may produce further feedback communicated to the various devices and therapy, monitoring, management elements hosted among or on such devices.

These features and attributes, and additional features and attributes, are described below in association with at least FIGS. 1-24.

At least some examples of the present disclosure are directed to a method and/or apparatus to perform patient care, patient management, and the like. In some examples, a method and/or system to facilitate patient care may comprise arrangements which provide for one or more of patient adherence (or patient compliance) prediction, non-adherence causation prediction, and non-adherence recommended action determination. In some examples, patient adherence refers to or includes a patient adhering to a treatment plan developed by a clinician. In some examples, non-adherence (or non-compliance), by contrast, refers to or includes patient deviations from a treatment plan. A non-adherent (or non-compliant) patient may include patients which are non-adherent and patients which may (technically) be adherent but which have issues. These issues may indicate that the patient may become or is predicted to become non-adherent, sometimes referred to as “predicted non-adherent” or “predicted or near non-adherent patient”). In some examples, a device manufacturer or device service provider may also communicate with the care team and/or the patient to facilitate patient care and clinician performance. In some examples, the device manufacturer or device service provider also may operate, supply, maintain a processing resource (e.g. cloud server) to provide an arrangement, framework, pathway, etc. by which patient management may be performed with related information being displayable on a user interface such as, but not limited to, a clinician portal.

It will be understood that the term clinician may refer to a device therapy technician, sleep study technician, a physician, or other medical worker (e.g. health care professional) suitably experienced to perform (or assist with) the example methods and systems of patient management and care of the present disclosure.

In some instances, the example methods and/or systems may comprise displaying at least some patient management information and tools (and/or device management information and tools) via a user interface (e.g. graphical user interface), such as on a desktop workstation, mobile computing tablet (or other convenient mobile computing device).

At least some examples of the present disclosure provide tools to obtain information about how and when a patient volitionally utilizes stimulation therapy based on, for example, the times and days that the patient turns the therapy on and off. This information provides objectivity regarding when a patient starts, pauses, and/or ends nightly therapy, among other information such as stimulation amplitude changes, and the like. In some examples, this objective information may be utilized to predict patient adherence and likely causes of non-adherence, and the like. By providing both a likelihood of adherence and likely cause(s) of non-adherence, a clinician may enhance their ability to discern relationships between stimulation therapy settings, volitional patient usage (or non-usage) of the stimulation therapy, patient symptoms, other comorbid disease/condition (e.g., sleep hygiene) and the like. The discernment of such relationships may, in turn, inform decisions resulting in changes to stimulation therapy parameters or resulting in no change to stimulation therapy parameters, changes or actions relating to other health parameters (e.g., sleep hygiene), etc., among other considerations.

Moreover, in some such examples, while the example display tools (e.g. available within a user interface) may inform the clinician in evaluating stimulation therapy treatment decisions, the example display tools still provide the clinician with appropriate autonomy in making medical decisions and/or using their discretion as appropriate regarding adjustment of parameters of the programmer, IMD, etc.

FIG. 1 is a block diagram schematically representing an example arrangement 100, deployable in an example method of (or as an example system for) patient management to facilitate patient care. As shown in FIG. 1, example arrangement 100 may comprise an array 102 of computing devices 110, each of which hosts a patient app 112 relating to patient care. The devices 110 may sometimes be referred to as patient devices, patient computing devices, and the like. The patient app 112 may provide patient education and/or enable communication with a caregiver, device servicer, device manufacturer, etc. In some examples, the patient app 112 may communicate patient usage information (and some related therapy metrics) to a cloud resource 135, a clinician device 150 (e.g., care entity), device servicer, device manufacturer, etc. In some such examples, at least some of the patient devices 110 may comprise a mobile computing device, such as a mobile phone, tablet, smartwatch, etc. which has a user interface to provide for operation of, and display of, the patient app 112. In at least some of the examples in which the patient device 112 may comprise a mobile computing device (e.g. mobile phone, tablet, etc.), the patient device 112 comprises a consumer electronic device (which is not a dedicated patient device and/or which is not a dedicated patient remote control (e.g. 120) but which may communicate with a dedicated patient device and/or dedicated patient remote control (e.g. 120).

As shown in FIG. 1, in some examples the example arrangement 100 may comprise an implantable medical device (IMD) 125. In some examples, the IMD 125 may be adapted for treating sleep disordered breathing (SDB) and/or other patient conditions (e.g. cardiac, pelvic disorders, etc.). A patient remote control 120 may communicate with the IMD 125 via a wireless communication protocol 126 either directly or indirectly via an intermediary communication element (e.g. antenna, other). In some examples, such wireless communication may take the form of inductive telemetry, Bluetooth, Bluetooth Low Energy (BLE), near infrared (NIF), near-filed protocols, Wi-Fi, Ultra-Wideband (UWB), and the like. In some examples, the IMD 125 may comprise a pulse generator, for example an implantable pulse generator (IPG), for generating stimulation therapy signals to be delivered to the patient via a stimulation element (e.g. electrode) within the patient.

In general terms, the patient remote control 120 enables a patient to have limited control over their stimulation therapy, such as turning the stimulation therapy on/off, pause, increasing or decreasing the amplitude of stimulation within a lower and upper limit set by a clinician (and/or device manufacturer, supplier, etc.), switching stimulation sites (with optional embodiments in which the IMD 125 is arranged to selectively deliver stimulation to two or more stimulation sites (e.g., sites along the same nerves, different nerves, etc.) as described below), etc. In some examples, the patient remote control 120 also tracks patient usage of these controls to enable a clinician, the patient, and others to learn about the patient's usage, therapy effectiveness, patient adherence, etc. In some examples, the patient remote control 120 also may receive some information from the IMD 125 regarding stimulation metrics, sensing metrics, etc.

In some examples, the patient remote control 120 is in communication with the patient app 112 such that patient app 112 on device 110 may receive the patient usage information from the patient remote control 120, as well as whatever therapy, sensing, etc. information was communicated from the IMD 125 to the patient remote control 120. In some examples, the communication between the patient remote control 120 and the patient app 112 (on patient device 110) may occur wirelessly 127 via a number of wireless communication protocols such as, but not limited to, a Bluetooth® wireless communication protocol. In some examples, the communication between patient remote control 120 and patient app 112 (on patient device 110) may occur via a wired connection. In other examples described below, the device 110, via the patient app 112, communicates directly with the IMD 125.

As noted here and elsewhere, the patient app 112 may communicate this information (received from the patient remote control 120) to one or more of the clinician devices 150 via resource 135 to facilitate patient management according to examples of the present disclosure. In some examples, the patient app 112 also may obtain some patient information through the patient's use of the app 112 which also may be communicated to the clinician devices 150 separately from, or integrated with, the patient usage information and therapy information from the patient remote control 120 and/or IMD 125. In some embodiments, the patient app 112 can be programmed to serve as a, or as the, patient remote control, providing the patient with any of the control features described above; in the descriptions below, therapy controls being effected by the patient via the patient remote control 120 are equally attributable to the patient app 112. For example, the patient app 112 can, via the patient device 110, directly communicate with the IMD (e.g., Bluetooth or other wireless communication protocol).

In general terms, the IMD 125 includes a pulse generator, an implantable pulse generator or implantable component of a pulse generator (collectively identified as “IPG”) 130 at least one stimulation component 131 and/or at least one sensing component 132. In some embodiments, an entirety of the IMD 125 is configured for implantation into the patient (e.g., a fully implantable IMD 125); in other embodiments, the IMD 125 can have a hybrid implantable configuration in which some components are implanted into the patient, and other components of the IMD 125 remain outside the patient (e.g., an internal lead and an external power source). With formats in which the IPG 130 is an implantable pulse generator, a power source (e.g., battery) is carried within a housing of the implantable pulse generator and from which stimulation energy is generated. With formats in which the IPG 130 is an implantable component of a pulse generator, the implantable component(s) can include a receiver unit (e.g., receiver coil or similar device) that receives a signal from an external device (external the patient) that typically would be positioned on top of the skin over the location of the receiver coil. The external device can generate/deliver the stimulation energy at desired settings (e.g., amplitude, pulse width, frequency, pulse train length, etc.) to be received by the implanted receiver unit and conducted to the stimulation component(s) 131 for activation of tissue. The implanted receiver unit may or may not be operable to modify the signal it receives prior to delivery to the stimulation component(s) 131. The external transmitter/controller may receive sensing signals from an external sensor(s), receive sensing signals from the implanted portion of the implantable component by telemetry, Bluetooth or other wireless communication protocol, etc. Unless stated otherwise, reference to “IPG 130” is inclusive of an external pulse generator, an implantable pulse generator and an implantable component of a pulse generator as described above.

In some examples, implanted portions may be sized and shaped for chronic implantation in head-and-neck region of the body. In some such examples, implanted portions may comprise or sometimes be referred to as a microstimulator. In some such examples, a microstimulator may comprise power (e.g. rechargeable, non-rechargeable), stimulation circuitry, and/or communication element).

In some examples, the stimulation component 131 comprises a stimulation engine to generate a stimulation signal to be applied to a tissue (e.g., nerve, muscle, etc.). In the examples, the tissue to be stimulated may comprise tissue to maintain or restore upper airway patency, such as but not limited to a hypoglossal nerve, an infrahyoid muscle (IHM)-innervating nerve (e.g., an ansa cervicalis-related nerve) and/or other nerves. In some such examples, the stimulation component 131 also may comprise circuitry for generating and delivering the stimulation signal. In some examples, the stimulation component 131 of the IMD 125 also may comprise a stimulation element, such as an electrode through which the stimulation signal may be applied to the target tissue. For example, the stimulation element can be electrically connected to the IPG 130 via a lead or the like). In other embodiments, the IPG 130 and the stimulation element can be provided as components of a single or integral device, such as a microstimulator. In some embodiments, the IMD 125 can include two or more stimulation elements deployed or implanted to stimulate multiple (i.e., two or more) stimulation sites. For example, a first one of the stimulation elements can be deployed or implanted to apply a first stimulation signal to a first target site and a second one of the stimulation elements can be deployed or implanted to apply a second stimulation signal to a second target site, with the first and second target sites being discrete or separate from one another. In one non-limiting example of a multiple stimulation site arrangement, the first stimulation element can be arranged to stimulate a hypoglossal nerve and the second stimulation element can be arranged to stimulate an IHM-innervating nerve. The IMD 125 can be programmed to operate (e.g., deliver stimulation energy to the stimulation component 131) in various manners. As used throughout the present disclosure, “device programming” is in reference to instructions acted upon by the IMD 125 that otherwise dictate or control the format (e.g., amplitude, pulse width, timing, etc.) of stimulation delivered to the patient.

In some examples, the sensing component 132 comprises a sensing engine to receive a sensing signal obtained relative to a tissue (e.g., muscle, organ, etc.). In the examples in which the IMD 125 comprises an IPG for treating sleep disordered breathing (SDB), the tissue to be sensed may be related to respiration, oxygenation, cardiac functions, upper airway patency, and the like. In some such examples, the sensing component 132 also may comprise circuitry for receiving and processing the sensing signal. In some examples, the sensing component 132 of the IMD 125 also may comprise a sensing element, such as an electrode or other element through which the sensing signal is obtained. In some examples, the sensing element may comprise an accelerometer for determining sleep information, respiratory information, posture information, and/or physical control information. The accelerometer may be implantable, and in some examples, may be incorporated within a device including a stimulation generating element (e.g., 131), such as an implantable pulse generator, which may comprise one example implementation of the implantable medical device 125.

Of course, in contexts in which the IMD 125 relates to bodily organs, functions, etc. other than sleep disordered breathing, the stimulation component 131 and sensing component 132 would be deployed relative to other tissues. For instance, the IMD 125 may be deployed to treat pelvic disorders, such as stress incontinence or other conditions, with applicable tissues including the bladder, pudendal nerve, urinary and/or anal sphincters and the like.

In some examples, the stimulation component 131 and/or sensing component 132 may be on-board the IMD 125, which in some examples may comprise a microstimulator.

In some examples, at least a portion of the stimulation component 131 and/or sensing component 132 may be separate from, and independent of, a housing of the IMD 125 with one or both components 131, 132 being in wired or wireless communication with the IMD 125.

As further shown in FIG. 1, the patient devices 110 may communicate with other devices, entities, etc. via a resource 135, such as a cloud resource, computer server, etc., via a wireless communication protocol as represented by directional arrows 140 and/or wired communication protocol in some examples. In some examples, information generated by, for example, the patient remote control 120 can be provided to the cloud resource 135 via the corresponding patient app 112. In some examples, the patient remote control 120 can communicate directly with the cloud resource 135. It will be understood that the cloud resource 135 may comprise a computing resource (including stored programming) provided via a third party to help provide and support patient management with the clinician devices 150 (e.g. clinician entities, care entities) and the patient app 112. In some examples, the cloud resource 135 may comprise at least a portion or, and/or an example implementation of, the control portion 1050 (FIG. 19) and user interface 1070 (FIG. 20). In some examples, the cloud resource 135 may be provided via a device manufacturer or servicer, or third party contracted by a device manufacturer. The cloud resource 135 may be hosted via the internet, World Wide Web, and/or other network communication link.

As further shown in FIG. 1, the example arrangement 100 may comprise one or more clinician devices 150, which provide care in some manner to a patient associated with one of the devices 110. Each clinician device 150 comprises a computing resource, such as workstation or other computing device which may be stationary or mobile, including a user interface to support operation and display of a portal 155 (e.g. a clinician portal). Among other functions and features, the portal 155 may comprise a patient management app 160 by which the particular care provider (e.g. medical clinic, sleep center, etc.) may manage patient care among a group of patients, for an individual patient, etc. The patient management app 160 also may enable communicating with other entities regarding patient care of the patients associated with devices 110. In some examples, the clinician device 150 and/or programmer 170 may provide the device programming to control, direct, etc., the IMD 125 assigned/implanted within a patient and/or obtain patient information and displaying such information to help manage patient care, etc. At least some aspects of patient management includes actions, tools, etc. to deliver stimulation therapy to a tissue of a patient, such as but not limited to tissue which may maintain or restore upper airway patency to treat sleep disordered breathing (SDB). In some examples, the example methods and/or systems of the present disclosure directed to patient management may lead to adjustments in stimulation programming (and related settings), which are used to configure the IMD 125 such that the stimulation programming settings become stored within, and are implemented via the IMD 125 to deliver stimulation therapy and related care to the patient. In some examples, the clinician device(s) 150 may communicate with the cloud resource 135 (e.g. network communication link, internet, web, etc.), as well as other devices of the arrangement (e.g., the patient devices 110, other clinician devices 150, etc.), via at least the cloud resource 135 as represented by indicator 165.

It will be further understood that the arrangement of the various portions displayed on each respective example user interface and/or the detailed listings within each respective displayed portion may form part of a method of patient management (including but not limited to stimulation therapy treatment) which a clinician (and/or other health care professional) may perform to advance patient care. Some example methods are described later in association with FIGS. 21-23. Similarly, it will be apparent from FIG. 1 that the various features, arrangements, components, etc. may be embodied as a system or apparatus.

In some examples, the example arrangement 100 may comprise a clinician programmer 170, which may periodically communicate with the IMD 125 wirelessly (e.g. inductive telemetry), as represented by indicator 175, to initially configure and/or modify the configured stimulation therapy settings, sensing settings, etc. of the IMD 125.

In some examples, the arrangement 100 may further include external monitoring circuitry 180 that senses or monitors patient data. The external monitoring circuitry 180 may comprise or form part of a device which is external to the patient. For example, the external monitoring circuitry 180 may be selectively worn by the patient such as around the wrist or on a finger, an arm, an ankle, a torso, a head, a neck, etc. of the patient. In some of these examples, external monitoring circuitry 180 may take the form of a wrist-watch, such as a smart watch. However, in some such examples, the external monitoring circuitry 180 may not be worn by the patient, but be positioned in a proximity to the patient sufficient for sensing to enable monitoring and/or diagnosis. In some of these examples, the external monitoring circuitry 180 may comprise a patient support such as, but not limited to, a mattress, bed, chair, and the like (e.g., a sleep tracking mat available from Withings).

In some examples, the external monitoring circuitry 180 may comprise some components worn by the patient and some components which placed in close proximity to, but not worn, by the patient.

Whether worn on the patient and/or positioned near the patient, sensing components associated with external monitoring circuitry 180 become oriented in sensing relation to one or more portions of the patient's body so as to facilitate sensing various physiologic phenomenon such as (but not limited to) the sensing parameters.

In some examples, the external monitoring circuitry 180 may communicate directly with patient device 110, such that the patient data sensed and/or determined by the external monitoring circuitry 180 may be selectively integrated directly into, and/or be complementarily combined with, SDB care-related information on the patient device 110, such as on patient app 112. This SDB care-related information may comprise patient management information and/or stimulation therapy-related information, as further described below, or other information.

In some examples, communication between the external monitoring circuitry 180 and the patient app 112 (hosted on patient device 110) may be performed via a wireless communication protocol (e.g., Bluetooth, infrared, near-field communication) and/or via a wired connection between the respective devices 110 and 180. In some instances, this communication pathway may sometimes be referred to as a direct communication pathway.

In some examples, the external monitoring circuitry 180 may comprise a general consumer product which is not specifically dedicated for use with patient communication device 110, patient remote 120, and/or IMD 125.

In some examples, the external monitoring circuitry 180 may be implemented according to at least some of substantially the same features and attributes as described by PCT Publication WO2022/182756 and/or the sensing functionalities described below. For example, the external monitoring circuitry 180 may sense various parameters, such as cardiac parameters (e.g., heart rate, an electrocardiogram (ECG), and/or other cardiovascular phenomenon), respiration, breathing rhythm, chest motion, oximetry, peripheral arterial signal, blood pressure, body position, snoring, AHI, time in bed, sleep time (e.g., start and stop times, total time, types of sleep), posture, among other physiologic parameters or environmental parameters (e.g., air temperature, humidity, amount of light, external interruptions). In some examples, the external monitoring circuitry 180 may sense a plurality of parameters using different sensing modalities, such as but not limited to an accelerometer, pressure sensor, acoustic sensor, light sensor and/or communication with other devices, such as smart thermostat, smart lights, and other devices, among other sensing modalities. In some examples, the external monitoring circuitry 180 may determine a parameter using a combination of the other parameters, such as determining sleep from a combination of posture, time, snoring, heart rate and/or other parameters. For example, the external monitoring circuitry 180 may determine whether snoring sounds are attributable to the patient or someone else, such as another human or animal.

The patient device 110 (e.g. phone) can provide sensing complementary with, or independent of, external monitoring circuitry 180. Nearable, instead of wearable. Among other sensed parameters: breathing/snoring from acoustic/sound sensing, breathing from visualized (camera) chest movement, ambient light, temperature, etc.

With the above in mind, the arrangement 100 of FIG. 1 further includes, in some embodiments, an adherence prediction engine 200. The adherence prediction engine 200 is generally programmed or formatted to evaluate a patient's usage of the IMD 125 and predict the patient's likelihood of adherence to a treatment plan going forward. In some examples, the adherence prediction engine 200 operates on, or is managed by, the cloud resource 135.

In some embodiments, the adherence prediction engine 200 is programmed to, or operates one or more protocols formatted to, review or consider usage of the IMD 125 by a particular patient (or “patient under review”) over a first period of time (or “Review Period”). The so-obtained information is compared with a database 202 of usage information obtained from a number of other patients, and from this comparison, a determination is made as to a likelihood of the patient under review adhering to the treatment plan for using the IMD 125. The database 202 can be considered part of the adherence prediction engine 200; in other embodiments, the database 202 is stored apart from, but accessible by, the adherence prediction engine 200.

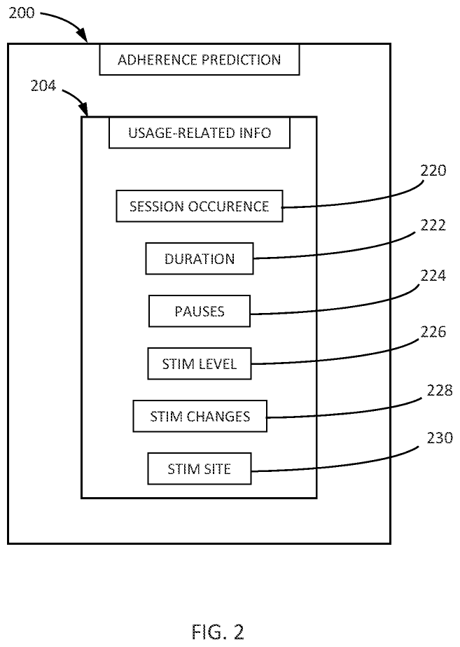

FIG. 2 is a block diagram of example usage-related inputs or information 204 provided to or considered by adherence prediction engine 200. As a point of reference, with embodiments in which the IMD 125 is intended to treat SDB, the IMD 125 can be programmed or otherwise adapted to implement a treatment plan by delivering stimulation energy at levels, as selected by a clinician, during a therapy session. The patient can be afforded limited control over their stimulation therapy, such as turning the stimulation therapy on/off, pause, and/or increasing or decreasing the amplitude of stimulation within a lower and upper limit set by a clinician (and/or device manufacturer, supplier, etc.), for example via the patient remote control 120. With this in mind, some the usage-related information 204 can include one or more of: therapy session occurrence 220, therapy session duration 222, therapy session pause 224, level(s) (e.g., amplitude) of stimulation delivered during a therapy session 226, changes to stimulation intensity 228, etc.

The usage information 204 can be obtained in various manners, for example by the adherence prediction engine 200 receiving corresponding information, indirectly (e.g., via the corresponding patient app 112) or directly, from the patient remote control 120 associated with the patient under review. The usage information 204 can include or implicate attributes of patient-initiated therapy sessions during which the IMD 125 operates to deliver stimulation energy to the patient (in accordance with instructions with programmed to the IMD 125) on a consistent or regular basis over an extended period of time (e.g., hours).

In some embodiments, an individual “therapy session” begins or is initiated when the patient enters or prompts a “therapy on”-type command (e.g., a command or prompt at the patient remote control 120 intended to activate the IMD 125 to deliver stimulation on a regular basis) and ends when the patient enters or prompts a “therapy off”-type command (e.g., a command or prompt at the patient remote control 120 intended to deactivate the IMD 125 or stop the IMD 125 from delivering stimulation for an extended period of time (e.g., hours)).

In some embodiments, once a “therapy off” command is received, the IMD 125 will not automatically “re-start” delivering stimulation energy at a later point in time; rather, a subsequent “therapy on” command or prompt from the patient must be received.

In some embodiments, a patient is afforded the ability to temporarily pause delivery of stimulation energy during the course of a particular therapy session, such as by the patient entering or prompting a “therapy pause”-type command at the patient remote control 120. Where the IMD 125 is arranged to selectively provide stimulation to two or more stimulation sites, a “therapy pause” command can be available for each of the stimulation sites independently and/or collectively. A “therapy pause” command temporarily pauses delivery of stimulation energy to the patient as part of a previously-initiated therapy session fora predetermined period of time (e.g., minutes). In some embodiments, the IMD 125 is programmed to automatically stop, then re-start, delivery of stimulation energy in response to a “therapy pause” command. Thus, during a particular therapy session (e.g., period of time from a “therapy on” command to a “therapy off” command), the patient may effect or enter zero “therapy pause” commands, a single “therapy pause” command, or multiple “therapy pause” commands. In some embodiments, programming for therapy session can establish and implement automated therapy session actions akin to one or more the therapy on, therapy off, and therapy pause commands. For example, programming can establish “Auto on”, “Auto off”, and “Auto pause” parameters, but afford the patient the ability to override and turn completely off. Auto on can be a selectable time (10 pm) or automatically initiate therapy upon automatically detecting sleep. A selectable delay (e.g., 30 min), after Auto on, can be established before stimulation actually starts. An Auto pause action can be triggered, for example, by a patient action such as sitting up or standing up, and staying in new position/posture (upright) for selectable period of time. Auto-resume then occurs when patient is detected or determined to have changed positions to a sleep (horizontal) position, which also may include delay feature (e.g., 15 min) before stimulation actually starts. These, and other, therapy on, therapy off, and therapy pause type information can be utilized with or as the therapy session occurrence information 220.

In some embodiments, a patient is afforded the ability to change an intensity of stimulation being delivered over the course of a particular therapy session, such as by the patient entering or prompting an “intensity change” command at the patient remote control 120. The “intensity change” command can include, for example, the patient affecting an increase or decrease in amplitude of the stimulation energy being delivered (e.g., within a lower and upper limit set by a clinician (and/or device manufacturer, supplier, etc.)).

With the above explanations in mind, the therapy session occurrence information 220 can include information relating to, or representative of, occurrences of individual therapy sessions, for example therapy session day/time. For example, the therapy occurrence information 220 can include or be representative of timestamped (i.e., calendar date and time) “therapy on”-type commands and timestamped “therapy off”-type commands performed by the patient. The timestamped “therapy on” and “therapy off” information can implicate that a therapy session has occurred (e.g., a timestamped “therapy on” command can designate that a particular therapy session began at the so-noted day/time; the subsequent timestamped “therapy off” command can designate the end of that particular therapy session). Further, a determined time difference between the timestamped “therapy on” command and “therapy off” command for a particular therapy session can be designated as the therapy session duration information 222 of that particular therapy session. The so-determined therapy session occurrence 220 and duration information 222 can be provided to the adherence prediction engine 200, the adherence prediction engine 200 can be programmed to determine therapy session occurrence 220 and duration 222 based upon raw data provided as the usage information 204, etc.

The therapy session pause information 224 can include information relating to or representative of patient-prompted pauses (if any) during a particular therapy session. For example, the therapy session pause information 224 can include or be representative of timestamped “pause”-type commands performed by the patient. The timestamped “pause” commands can be correlated with the date/duration of a particular therapy session as described above. In some embodiments, the duration of a pause in the delivery of stimulation energy in response to a received “pause” command is predetermined and thus known. In some embodiments, for example where the patient is afforded the ability to select a duration of the pause in stimulation delivery as part of a “pause” command, the therapy session pause information 224 can further include the pause duration as selected by the patient in connection with a particular therapy pause request. Regardless, the so-correlated therapy session pause information 224 (e.g., each “pause” command is tied, based on day/time, to a particular therapy session) can be provided to the adherence prediction engine 200, the adherence prediction engine 200 can be programmed to tie “pause” commands with particular therapy sessions based upon raw data provided as the usage information 204, etc.

The therapy session stimulation level information 226 can relate to one or more parameters that characterize the level or intensity of stimulation energy delivered to the patient during a particular therapy session. In some embodiments, the therapy session stimulation level information 226 can include one or more of a maximum amplitude of stimulation energy delivered during a particular therapy session, normalized amplitude of stimulation energy delivered during a particular therapy session, etc. The therapy session stimulation level information 226 can be provided to the adherence prediction engine 200 (e.g., tied, based on day/time, to a particular therapy session), the adherence prediction engine 200 can be programmed to tie received stimulation level information 226 with particular therapy sessions based upon raw data provided as the usage information 204, etc.

The changes to stimulation intensity information 228 can include information relating to or representative of patient-prompted intensity changes (if any) during a particular therapy session. For example, the changes to stimulation intensity information 228 can include or be representative of timestamped “intensity change”-type commands performed by the patient. The timestamped “intensity change” commands can be correlated with the date/duration of a particular therapy session as described above. In some embodiments, the information relating to or representative of patient-prompted intensity change can further include a parameter or value indicative of stimulation intensity resulting from a particular “intensity change” command. For example, the changes to stimulation intensity information 228 can include a maximum amplitude of stimulation energy. Thus, changes to stimulation intensity information 228 can include maximum amplitude of stimulation energy being delivered during a particular therapy session before and after an “intensity change” command is prompted by the patient. Regardless, the so-correlated intensity change information (e.g., each “intensity change” command is tied, based on day/time, to a particular therapy session, as are any recorded stimulation energy parameters, such as maximum amplitude) can be provided to the adherence prediction engine 200, the adherence prediction engine 200 can be programmed to tie “intensity change” commands and optionally one or more corresponding parameter values (e.g., maximum amplitude following the intensity change) with particular therapy sessions based upon raw data provided as the usage information 204, etc.

With non-limiting examples in which IMD 125 for the patient under review is arranged to selectively stimulate two (or more) target sites (e.g., the hypoglossal nerve and an IHM-innervating nerve), the usage information 204 will include or designate independent usage of each of the different available stimulation sites and programming per stimulation site. With these and other embodiments, the therapy session occurrence 220, therapy session duration 222, therapy session pause 224, level(s) (e.g., amplitude) of stimulation delivered during a therapy session 226, changes to stimulation intensity 228, etc., data as provided to, or determined by, the adherence prediction engine 200 can be demarcated into, or by, each of the stimulation sites of the patient under review (designated by “stim site” 230 in FIG. 2). For example, the therapy prediction engine 200 can determine or predict patient adherence with respect to one or more or all stimulation target sites individually, adherence with respect to all stimulation target sites collective, or both.

In some embodiments, the usage information 204, as delivered to and/or as acted upon by the adherence prediction engine 200 to determine or predict a likelihood of patient adherence to a treatment plan, need not include, or otherwise omits, one or more of the parameters 220-230. In some embodiments, the usage information 204, as delivered to and/or as acted upon by the adherence prediction engine 200 to determine or predict a likelihood of patient adherence to a treatment plan, includes one or more other parameters in addition to, or in place of, one or more of the parameters 220-230. In some embodiments, the adherence prediction engine 200 can derive other usage information of interest from the usage information 204. For example, from the therapy occurrence information 220, the adherence prediction engine 200 is apprised as to the days on which stimulation therapy was performed. From this same information, the adherence prediction engine 200 can determine days on which no stimulation therapy was performed. The day(s) of non-use can be a useful parameter for predicting likelihood of patient adherence (and/or declaring or characterizing a patient as non-adherent) in some embodiments. Alternatively, the day(s) of non-use can be separately determined and provided to the adherence prediction engine 200 as part of the usage information 204.

Regardless of the content and/or format of the usage information 204, the adherence prediction engine 200 can be programmed to, or can operate one or more protocols formatted to, determine or predict a likelihood of patient adherence to a treatment plan based upon a comparison of the usage information 204 with the database 202 of usage information obtained for a plurality of other patients. In some embodiments, the database 202 provides or accumulates previous IMD usage patterns for patients under circumstances generally similar to the patient under review (e.g., the previous usage patterns of the database 202 are for previous patients using an IMD to treat a malady (e.g., SDB) similar to the patient under review according to a treatment plan similar to the patient under review). The previous usage patterns of the database 202 can include usage patterns of patients that were determined or deemed to be adherent to a treatment plan, and patients that were determined or deemed to be non-adherent to a treatment plan. In some embodiments, the database 202 includes previous usage patterns for at least five hundred previous patients; optionally at least one thousand previous patients. In some embodiments, the adherence prediction engine 200 utilizes or incorporates machine learning, for example a pre-trained deep learning model, to predict a likelihood of adherence for a patient under review when comparing the usage information 204 of the patient under review with the previous usage patterns of the database 202. With these and related embodiments, the database 202 can aggregate the previous usage patterns with determined therapy trajectories as a pre-trained deep learning model to which the usage information 204 of the patient under review is matched. The deep learning model can comprises models such as, but not limited to, convolutional networks (e.g., deep belief, neural), belief networks, Boltzmann machines, deep coding networks, stacked auto-encoders, stacking networks (e.g., deep or tensor seep), hierarchical-deep models, deep kernel machines, and the like. Other machine learning model approaches can also be used, such as an artificial neural network, support vector machine (SVM), etc.

In some embodiments, the adherence prediction engine 200 is programmed to (or operates one or more protocols formatted to) determine or predict a likelihood of patient adherence upon completion of a Review Period. In some embodiments, the Review Period can be a certain number of days (e.g., 7 days, 14 days, 30 days, 60 days, 90 days, etc.) following occurrence of a specified or pre-determined event, such as initial, normal use of the IMD 125 by the patient under review, a change in treatment plan, etc. By way of non-limiting example, a Review Period can begin after the IMD 125 has been implanted and programmed, and the patient under review is instructed to begin the treatment plan by using the IMD 125 on a regular basis. In another example, a Review Period can begin when the patient under review has been using the IMD 125 according to a first treatment plan in which the IMD 125 is programmed to not exceed a first maximum stimulation amplitude, and then a second treatment plan is implemented (for example, following a polysomnography (PSG) study whereby the IMD 125 is programmed to not exceed a second maximum stimulation amplitude differing from the first maximum stimulation amplitude. With these and related embodiments, at least some of the previous usage patterns of the database 202 entail previous usage patterns that include occurrence of the same, specified event and extend over a time period longer than the Review Period.

In some non-limiting examples, the adherence prediction engine 200 is programmed to, or operates one or more protocols formatted to, recognize or identify relationships or correspondences of the usage information 204 of the Review Period with a segment of one or more previous usage patterns, or a deep learning model, of the database 202 over a period of time corresponding with the Review Period (e.g., previous usage pattern segment over a period of time similar or identical to the Review Period, previous usage pattern segment over a period of time starting from occurrence of a specified or pre-determined event similar or identical to the specified or pre-determined event of the Review Period, etc.). For ease of explanation, any previous usage pattern, or model, of the database 202 having a usage pattern segment recognized or identified as being similar to the usage information 204 of the patient under review can be termed a “Predictive Previous Usage Pattern”, and the period of time of the Predictive Previous Usage Pattern otherwise corresponding with the Review Period can be termed a “Selected Period”. Those portions of an identified Predictive Previous Usage Pattern immediately following the Selected Period can be termed a “Predictive Therapy Trajectory”. One or more Predictive Therapy Trajectories can be reviewed or evaluated to determine the corresponding previous patient's long term adherence to a treatment plan. The Predictive Therapy Trajectory or Trajectories can be used to implicate or inform a likelihood of the patient under review adhering to the treatment plan. For example, where the previous patient of an identified Predictive Previous Usage Pattern is deemed to be highly adherent to a treatment plan, it can be considered that the patient under review is more likely to adhere to his/her treatment plan. Conversely, where the previous patient of an identified Predictive Previous Usage Pattern is deemed to be non-adherent to a treatment plan, it can be considered that the patient under review is less likely to adhere to his/her treatment plan.

Similarity between the usage information 204 of the patient under review and the usage pattern segment of an identified Predictive Previous Usage Pattern can be considered or accounted for when determining or designating the relevancy of the corresponding Predictive Therapy Trajectory to the likelihood of adherence for the patient under review. Where multiple Predictive Previous Usage Patterns are identified, the weight or relevancy of each so-identified Predictive Previous Usage Pattern to the likelihood of adherence for the patient under review can consider or account for a determined level of similarity with the usage information 204. Moreover, these and other factors can be considered by the adherence prediction engine 200 in determining a confidence level for a resulting adherence prediction.

In some embodiments, a likelihood of patient adherence designation generated by the adherence prediction engine 200 implicates or predicts whether the patient under review will have a high or low daily usage, on average, of the IMD 125. In some embodiments, a “high daily usage” is more than four hours, and a “low daily usage” is less than four hours. Other daily usage designations or values can also be employed. For example, high daily usage can be more than five days per week, and low daily usage is less than five days per week. In other examples, high daily usage is the combination of more than five nights per week of four hours use per night, and low daily usage is designated as less than four hours of use on fewer than five nights per week. The likelihood of patient adherence designation or classification generated by the adherence prediction engine 200 can alternatively or additionally take other forms. For example, the likelihood of patient adherence designation or classification can be either an “adherent” or a “non-adherent” prediction (optionally along with a probability score). In another example, the likelihood of patient adherence designation or classification can be or include an assigned descriptor or descriptive word such as “poor”, “moderate” and/or “good”; etc.

In some embodiments, the adherence prediction engine 200 is operated to determine or predict a likelihood of patient adherence, for example a prediction of whether the patient under review will have a high daily usage or a low daily usage, for a patient at the beginning of a treatment plan. Commensurate with the above explanations, following initial implantation and programming of the IMD 125, the patient is instructed to begin using the IMD 125 in accordance with the treatment plan, for example on a nightly (or daily) basis. It can be helpful to evaluate the patient's usage shortly after this initial phase of use. High early usage can be a useful predictor of continued usage or adherence. With some patients; however, usage rapidly drops in the first 90 days and can be a useful predictor of longer term non-adherence. For example, it has surprisingly been found that a viable prediction of likely adherence can be made shortly after the patient begins the treatment plan (e.g., 90 days or less, optionally 30 days or less); under circumstances where a particular patient is predicted to have lower-than-desired adherence shortly after the treatment program begins, corrective actions can be taken to improve the patient's adherence.

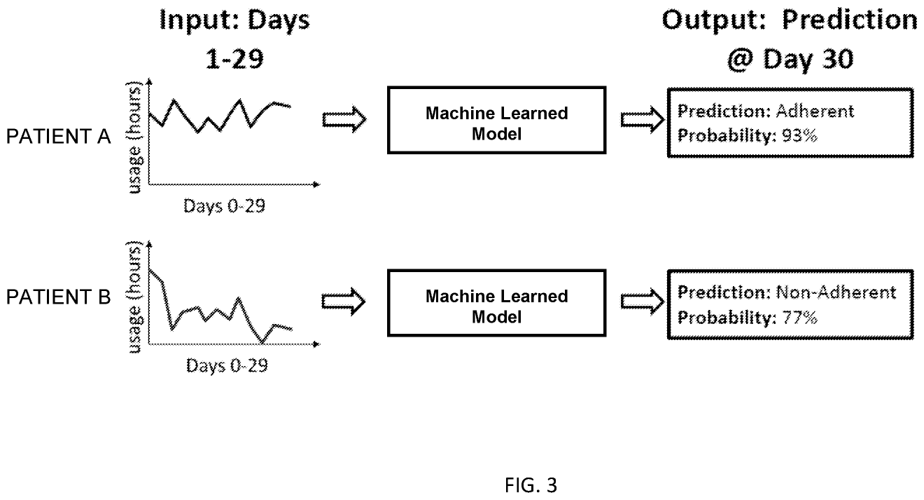

With the above in mind, in some embodiments the adherence prediction engine 200 is programmed or operates to determine or predict a likelihood of adherence based on a Review Period of 90 days, 30 days, 14 days, etc., starting with the day the patient is instructed to begin the treatment plan. The usage information 204 is collected over the Review Period, and provided to the adherence prediction engine 200. The Review Period usage information 204 is compared with the prior usage patterns and/or model(s) of the database 202. For example, the Review Period usage information 204 can be considered or treated as an input pattern that is matched to the prior usage patterns and/or model(s) as described above. From the Predictive Therapy Trajectory of identified Previous Usage Pattern(s), a determination of a likelihood of the patient adhering to the treatment plan is generated. For example, the adherence prediction engine 200 can, based on less than 90 days of usage information, output an individualized prediction of high daily usage or low daily usage, on average, after the first 90 days (or three months) of therapy. Alternatively or in addition, the adherence prediction engine 200 can, based on less than 90 days of usage information, output a probability of high daily usage or low daily usage, on average, on the first 90 days (or three months) of therapy. In other embodiments, the adherence prediction engine 200 can operate to predict or estimate a likelihood of the patient to adhere to the treatment plan for some other future time frame (e.g., 90 days into the future, 120 days into the future, etc.). Non-limiting examples of individualized predictions that can be generated by the adherence prediction engine 200 for two different patients (Patient A and Patient B) based on 30 days of usage information is provided in FIG. 3.

Returning to FIG. 1, the adherence prediction engine 200 can operate to generate adherence predictions for a number of other Review Periods. Features of the present disclosure are in no way limited to predicting patient adherence following the initial phase of use. Useful adherence predictions can be generated at any point in time and for any reason, such as (but not limited to) during or following a therapy maintenance stage of use, during or following a therapy adjustment phase of use, etc. The determinations or predictions generated by the adherence prediction engine 200 can be based on one or more of: the number of days the patient did not perform a therapy session over the course of the Review Period, the duration of each therapy session over the course of the Review Period, the number of therapy sessions with a duration of more than 4 hours and/or the number of therapy sessions with a duration of less than 4 hours over the course of the Review Period, the number of pauses in each therapy session over the course of the Review Period, the intensity of stimulation energy delivered during each of the therapy sessions over the course of the Review Period, the maximum amplitude of stimulation energy delivered during each of the therapy sessions over the course of the Review Period, the changes in stimulation intensity effected by the patient over the course of the Review Period, etc.

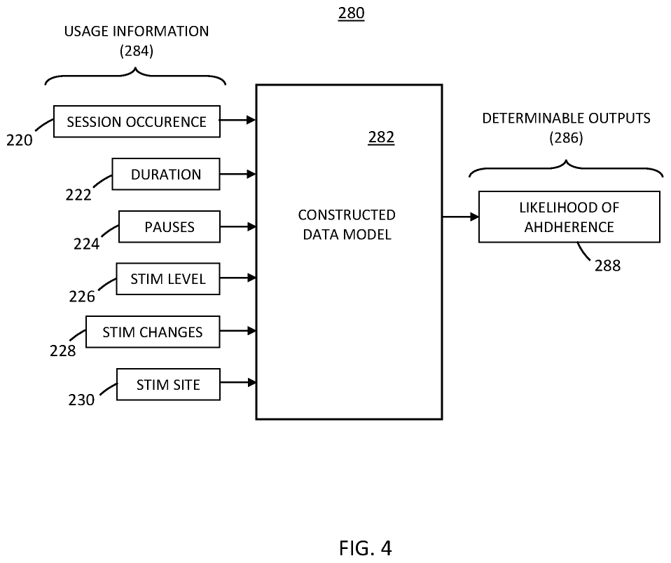

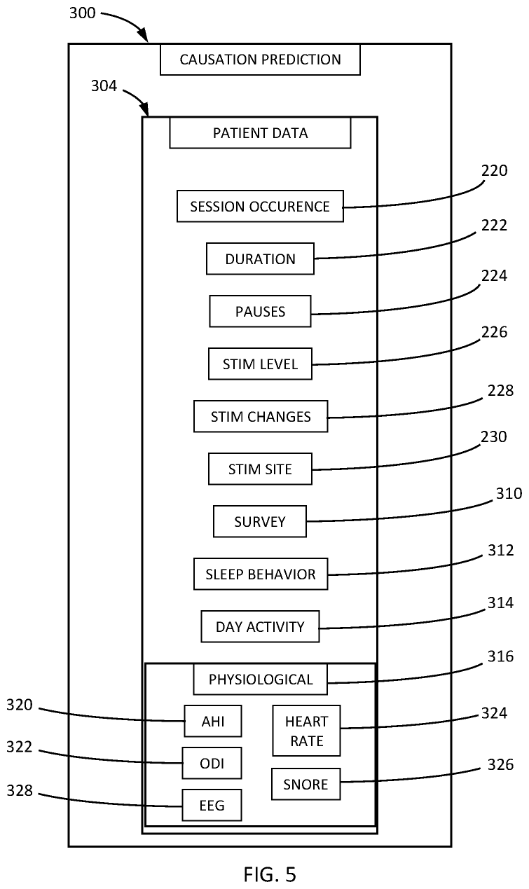

FIG. 4 is a diagram schematically representing an example method 260 of using a constructed data model 282 for performing an adherence prediction, such as via usage information 284. As shown in FIG. 4, the usage information 284 are fed into the constructed data model 282 (e.g., pre-trained deep learning model), which then produces a determinable output 286, such as a likelihood of adherence 288, which is based on the usage information 284. In some examples, the usage information 284 are obtained via at least the patient app (e.g., 112 in FIG. 1) and/or a patient remote (e.g., 120) utilized by the patient under review.

With reference to FIG. 1, the arrangement 100 can further include a causation prediction engine 300. In general terms, the causation prediction engine 300 is programmed to, or operates one or more protocols formatted to, identify likely reasons or causes of non-adherence for a patient exhibiting, or predicted to have, lower-than-desired adherence to a treatment plan. The causation prediction engine 300 can be prompted to perform a causation review under various circumstance, for example in response to the adherence prediction engine 200 predicting or determining that the patient under review has a low or lower likelihood of adhering to the treatment plan. Alternatively or in addition, the causation prediction engine 300 can perform a causation review in tandem with operation of the adherence prediction engine 200 to predict patient adherence as described above. Alternatively or in addition, the causation prediction engine 300 can perform a causation review independent of the adherence prediction engine 200. Thus, in some embodiments, arrangements of the present disclosure need not include the adherence prediction engine 200. In some examples, the causation prediction engine 300 operates on, or is managed by, the cloud resource 135.

In some embodiments, the causation prediction engine 300 is programmed to, or operates one or more protocols formatted to, review or consider a patient's usage of the IMD 125 and optionally additional parameters over a first period of time (or Review Period). FIG. 5 is a block diagram of example inputs or patient data 304 provided to or considered by the causation prediction engine 200. The patient data 304 can include one or more of the therapy session occurrence 220, therapy session duration 222, therapy session pause 224, level(s) (e.g., amplitude) of stimulation delivered during a therapy session 226, changes to stimulation intensity 228 (one or more of which can be demarcated by the particular stimulation site 230) as described above.

The patient data 304 can optionally further include patient survey information 310. In some examples, the example patient survey information 310 can be controlled or generated by a patient survey engine that may comprise a portion of, and/or be implemented via, control portion 1050 in FIG. 18. In general terms, the patient survey engine controls and supports operation of a patient survey displayed on a user interface, such as via an app (e.g., 112 in FIG. 1) on a display screen of a computing device, which may be mobile such as a mobile phone, tablet, etc. (e.g., 110 in FIG. 1). However, it will be understood an example patient survey may be displayed and provided on a user interface of a stationary computing device, in some examples.

In some examples the patient survey information 310 may comprise answers to one or more queries posed to a patient regarding: (A) nightly usage (“are you using the device every night?); (B) comfort (“is the stimulation comfortable?”); (C) hindrances; (D) device visible indicators; (E) snoring (“has your snoring improved?”); (F) feeling (e.g. “do you feel tired?”); and/or other parameters (e.g., where the IMD 125 as arranged to the patient provides for selective stimulation at two or more different stimulation sites, questions can be posed as to usage, comfort, etc., for each available stimulation site).

In some examples, hindrances may relate to factors which are preventing (or perceived to be preventing) a patient from employing therapy every night. At least some example hindrance factors may comprise: (1) it is uncomfortable; (2) it wakes me up; (3) I forget to use it every night, and/or (4) other reasons.

It will be understood that such example patient surveys are not limited to the exact words noted above and may use different expression, terms, etc. while still seeking and obtaining the same type of information. Moreover, the example patient survey information 310 is not strictly limited to the example queries described above. Any type of patient reported survey/questionnaire can be used as an input for the patient survey information 310 (e.g., a personality type questionnaire). The patient survey information 310 can be provided to the causation prediction engine 300 (e.g., tied to a particular day/time, optionally tied to a particular therapy session), the causation prediction engine 300 can be programmed to tie received patient survey information with a particular day/time and/or particular therapy session based upon raw data provided as the patient survey information 310, etc.

The patient data 304 can optionally further include sleep behavior-related parameter 312. The sleep behavior-related parameter 312 is indicative of a quality of sleep experienced by the patient, and can be based upon or generated by one or more sensor-based devices associated with the patient. For example, a useful sensor device can be a bed sensor or external sleep mat, and that can generate information or data indicative of sleep behaviors or patterns (e.g., time the patient first lays in bed, length of time in bed, movement during the night, movement during sleep, movement during delivery of stimulation therapy, in/out of bed during the night, etc.). Other sensor formats are also acceptable.

While the sleep behavior-related sensor parameters 312 can be provided to the causation prediction engine 300 as a determined parameter (e.g., a rated sleep behavior parameter tied to a particular day/time, optionally tied to a particular therapy session), the causation prediction engine 300 can be programmed to determine a sleep behavior-related parameter from raw sensor data provided as the information 304 and tie the so-determined parameter with a particular day/time and/or particular therapy session, etc.

The patient data 304 can optionally further include daytime activity parameters 314. The daytime activity parameter 314 is indicative of a patient's level of activity when awake, and can be based upon or generated by one or more sensor-based devices associated with the patient. For example, a useful sensor device can be an accelerometer worn by and/or implanted within the patient, and that can generate information or data indicative of movement. Other sensor formats are also acceptable.

While the daytime activity parameters 314 can be provided to the causation prediction engine 300 as a determined parameter (e.g., a rated daytime activity parameter tied to a particular day/time, optionally tied to a particular therapy session), the causation prediction engine 300 can be programmed to determine a daytime activity parameter from raw sensor data provided as the patient data 304 and tie the so-determined parameter with a particular day/time and/or particular therapy session, etc.

The patient data 304 can optionally further include one or more physiological parameters 316. The physiological parameters 316 can include Apnea-Hypopnea Index (AHI) parameters 320, Oxygen Desaturation Index (ODI) parameters 322, heart rate parameters 324, snoring parameters 326, electroencephalogram (EEG) parameters 328, etc. Each of the physiological parameters 320-328 can be based upon information or data provided by one or more sensors associated with the patient under review. In some examples, at least one of the sensors may be external to the patient, such as a wearable sensor/device or part of a home monitoring system. In some examples, at least one of the sensors may be implantable, and may further comprise a portion of another implantable medical device (e.g., 125 in FIG. 1) such as an implantable pulse generator. In some examples, at least one sensor may comprise an accelerometer, which may be wearable or implantable (e.g., within or on the implantable pulse generator).

The physiological parameters 316 can each be provided to the causation prediction engine 300 as a determined parameter (e.g., a determined AHI value or rating tied to a particular day/time, optionally tied to a particular therapy session). The causation prediction engine 300 can be programmed to determine a physiological parameter rating or value from raw sensor data provided as the patient data 304 and tie the so-determined parameter with a particular day/time and/or particular therapy session, etc. For example, AHI can be considered the number of times an apnea or hypopnea event occurs during the night, divided by the number of hours of sleep. In some examples, AHI can be the number of apnea or hypopnea events per hour, computed throughout the treatment period. As a point of reference, a patient's AHI may vary hour-by-hour (or smaller time increments) dependent on their position (supine vs. side) or other factors. A single/overall night time average might obscure valuable information versus if/when AHI calculated hour-by-hour, minute, etc. When calculated more frequently, used to adjust therapy parameters, stimulation target, sensing modalities/thresholds, etc., during the therapy session, perhaps in real time or at least much more frequently than adjust once/day. The so-determined AHI value or rating for a particular day/night and/or at various points in time throughout a particular day/night can be determined based upon sensor information and can be provided to the causation prediction engine 300 (e.g., the AHI parameter 320) as the determined AHI value or rating and tied to the particular day/night.

One or more of the ODI parameters 322, heart rate parameters 324, snoring parameters 326 (e.g., number of minutes snoring, number of snoring occurrences per hour, etc.), and/or EEG parameters 328 can be similarly generated and provided to the causation prediction engine 300. Other sleep apnea severity parameters or values can also be provided, such as patient-reported measurement of sleepiness such as the Epworth Sleepiness Scale, patient-reported measurement of daytime activity such as the Functional Outcomes of Sleep Questionnaire, etc. Sleep quality metrics can be provided to or determined by the causation prediction engine 300. In some examples, the determined parameters may comprise various parameter for quantifying aspects of sleep (e.g., sleep quality) such as, but not limited to, sleep state, sleep stage, time, score, and time-in-bed. In some such examples, the sleep state parameter may comprise a sleep-wake status, sleep start time, sleep stop time, sleep pause times, and the like. The time parameter may relate to total sleep time, total stimulation therapy time, and/or other time parameters related to sleep. In some examples, the score parameter may relate to a sleep score which provides information about sleep quality. In some examples, the sleep score may be impacted by external interruptions, such as noises and/or movement that occur during the total sleep time. In some examples, the time-in-bed parameter may provide a total time-in-bed within an intended sleep period (e.g. 10 pm to 6 am), within a twenty-four hour period, etc. Among other aspects, the time-in-bed parameter may help provide context for the start times, stop times, number and duration of pause times, and/or total therapy-on duration, which in turn may be used to evaluate therapy efficacy, patient adherence, and related care parameters.

Regardless of the content and/or format of the patient data 304, the causation prediction engine 300 can be programmed to or can operate one or more protocols formatted to, identify or predict the likely cause (or causes) of non-adherence to a treatment plan based upon a review or analysis of the patient data 304 collected over a designated Review Period. The review or analysis can take various forms, some examples of which are provided below.

In some embodiments, the causation prediction engine 300 is programmed to include (or operates one or more protocols that provide) several pre-determined or recognized causes or categories of patient non-adherence relevant to the particular malady being treated in the patient under review. For example, where the patient under review is being treated for SDB, some recognized common causes of patient non-adherence can include patient sleep issues or habits, stimulation-related discomfort, and efficacy of treatment. Recognized patient sleep issues or habits can include insomnia, irregular timing of sleep, and irregular length of sleep. Other recognized or hypothesized causes or categories can also be incorporated. For example, upper airway collapsibility can implicate SDB and can be assessed in various manners/via various metrics such as average continuous positive airway pressure (CPAP), polysomnography (PSG) signal shapes and metrics, airway imaging and metrics, anthropomorphic parameters (e.g., neck size, hip/waist ratio), etc. Regardless, the causation prediction engine 300 can be provided with, or programmed to determine, predictors or characteristics, and their relationships to one another, of each of the recognized causes. The predictors or characteristics, in turn, can be based upon or generated by data features expected to be collected for the patient under review. In some embodiments, the predictors or characteristics, along with their relationships, can be based upon a statistical review of the features derived from previous patient datasets. For example, various datasets for the previous patients (e.g., the database 202 (FIG. 1)) can be collected over the course of a designated time period (e.g., 90 days), and from these datasets, various features extracted and relationships to recognized causes categorized. A wide variety of other analyses are also acceptable.

Regardless of how the recognized causes and predictors are derived and categorized, the collected patient data 304 can be applied to or compared with the predictors in arriving at an identified or predicted cause(s) of non-adherence in the patient under review. In this regard, the features extracted from the collected patient data 304 can correspond with the some or all of the predictors recognized, or operated, by the causation prediction engine 300. In the context of SDB treatment, some extracted features can include, but are not limited to: therapy session stimulation amplitude (e.g., normalized amplitude), days of non-use, therapy session duration, therapy session pauses (number and/or duration), therapy session start time (e.g., relative to midnight or other time of day reference), and therapy session end time (e.g., relative to midnight or other time of day reference). Where the IMD 125 as arranged to the patient provides for selective stimulation at two or more different stimulation sites, extracted features can be designated and reviewed relative to each available stimulation site. The number of extracted features can be used to inform the number of predictors or groups (e.g., clusters) for evaluation. By way of non-limiting example, applying a Gaussian Mixture Model clustering method to fifteen extracted features, Bayesian Information Criterion informs to apply or use seven clusters. Other statistical analyses or techniques can alternatively be employed. For example, a data model can be constructed to identify the cause(s) of non-adherence via the collected patient data 304. In some examples, the data model can be constructed via training the data model (e.g., akin to the model of FIG. 4, but implemented to determine likely/predicted cause of non-adherence). In some examples, the data model can comprise at least one data model type that includes a machine learning model, which may comprise an artificial neural network, support vector machine (SVM), deep learning, clustering, etc.

In some examples in which the data model may comprise a clustering method(s), the clustering method(s) may comprise hierarchical clustering, k-means clustering, density-based clustering, and the like. In some examples, the hierarchical clustering can be used to construct a hierarchy of clusters of patient data. In some such examples, the hierarchical clustering utilizes can employ a “bottom up” approach (e.g. agglomerative) wherein each data point starts in its own cluster, and pairs of clusters are merged at progressively higher levels of the hierarchy. However, in some examples, the hierarchical clustering utilizes a top-down approach in which all data points start in one cluster, and then clusters are split at progressively lower levels of the hierarchy.

In some examples, the k-means clustering implementation may comprise placing the collected patient data into k clusters, where k is an integer equal or greater than two. Via such clustering, each data point belongs to a cluster having a mean that is closer to the data point than any means of the other clusters. However, in some examples, a machine learning model (MLM) may comprise density-based clustering, which may be used to group together physiological data points that are close to one another, while identifying as outliers any data points that are far away from other data points.

From the above evaluations, the causation prediction engine 300 can generate or output a predicted non-adherence cause. In some examples, the evaluations performed by the causation prediction engine 300 may result in the identification of two (or more) potential causes of non-adherence in the patient under review. With these and related embodiments, the causation prediction engine 300 can be programmed to, or can operate one or more protocols formatted to, prioritize or weight the two (or more) identified potential causes of non-adherence. In some examples, prioritization can be based upon a determined standard deviation in two or more of the features extracted from the patient data 304 and, optionally, a weight or importance of the so-reviewed features relative to the corresponding potential cause of non-adherence. The extracted feature of the patient data 304 found to have the greatest standard deviation (over the Review Period) can be designated has having priority over other extracted features; this weighting, in turn, can dictate which of the two (or more) identified potential causes of non-adherence is deemed to likely be more relevant to the patient under review. In the context of SDB, some non-limiting examples of features useful for prioritizing between two (or more) identified causes of non-adherence include: time of day of therapy off commands, number of therapy sessions of less than one hour, number of therapy pause commands, number of missed days, duration of therapy session, total therapy off time, total therapy on time, and duration of sleep.

In addition or as alternative to the above techniques, the causation prediction engine 300 can be programmed to (or can operate one or more protocols formatted to) identify situations in which the patient under review is utilizing stimulation levels likely to lead to non-adherence (“Therapy Discomfort Identification”). As a point of reference, with some SDB treatment plans, a patient is afforded the ability to change an intensity of stimulation (e.g., within a lower and upper limit set, for example, by a clinician) being delivered over the course of a particular therapy session and/or to select a stimulation intensity (e.g., within a lower and upper limit set, for example, by a clinician) at the start of a particular therapy setting. Given this ability, some patients are inclined to self-increase stimulation intensity often. Further, with some of these same SDB treatment plans, the patient is asked or encouraged to attempt to use an increased stimulation intensity (e.g., amplitude) over the course of several therapy sessions (e.g., during the initial phase of use, the patient can be asked to increase intensity over the course of the first ninety days). However, the increased intensity may not have a substantive therapeutic benefit. In other examples, a patient progressively or periodically increasing stimulation intensity over the course of multiple therapy sessions may experience a beneficial therapeutic effect for the first several increases, but then no or minimal therapeutic benefit from even further increases. Moreover, some patients are more sensitive to stimulation than others. For these and other patients, increased stimulation intensity may negatively impact later or long-term usage (e.g., lead to non-adherence). Where the IMD 125 as arranged to the patient provides for selective stimulation at two or more different stimulation sites, some patients may experience more discomfort at one stimulation site versus another.

With the above in mind, in some embodiments the causation prediction engine 300 is programmed to (or operates one or more protocols formatted to) identify stimulation-caused patient discomfort as a cause of actual or potential patient non-adherence based on comparisons or relationships between one or more therapy performance parameters and stimulation intensity over time. For example, the patient data 304 collected over the course of a designated Review Period can be reviewed and a sleep apnea severity value or parameter indicative of therapy performance obtained or determined for at least some of the therapy sessions of the Review Period, along with a parameter representative of stimulation intensity for the same therapy sessions.

With examples in which the patient under review is being treated for SDB, a useful sleep apnea severity value or therapy performance parameter is an Apnea Hypopnea Index (AHI) value. Other useful sleep apnea severity values or parameters include Oxygen Desaturation Index (ODI), number of minutes snoring, number of snorting occurrences per hour, patient-reported measurement of sleepiness (e.g., Epworth Sleepiness Scale), patient-reported measurement of daytime activity (e.g., Functional Outcomes of Sleep Questionnaire), etc. In some examples, then, the causation prediction engine 300 is informed of, or determines, the sleep apnea severity value for at least some of the therapy sessions of the Review Period. Another useful therapy performance parameter is therapy session duration. In some examples, then, the causation prediction engine 300 is informed of, or determines, a duration 222 of at least some of the therapy sessions of the Review Period (e.g., duration from “therapy on” command to “therapy off” command, minus the duration of any “therapy pause” commands). Other therapy performance parameters can alternatively be utilized.

A value representative of stimulation intensity from at least some of the therapy sessions of the Review Period is also obtained or determined. Some useful parameters representative of therapy session stimulation intensity are the maximum stimulation amplitude utilized during a therapy session, normalized stimulation amplitude of a therapy session, etc. Where the IMD 125 as arranged to the patient provides for selective stimulation at two or more different stimulation sites, stimulation intensity can be considered or evaluated on a per stimulation site basis. Regardless, the causation prediction engine 300 ties or links the obtained stimulation intensity value with the obtained therapy performance value from the same therapy session.

The causation prediction engine 300 tracks and correlates the paired therapy performance/stimulation intensity values over the Review Period. From this correlation, a potential patient discomfort situation can be identified. For example, the causation prediction engine 300 can operate to designate occurrence of a negative correlation under circumstances where an increase in maximum stimulation amplitude does not provide a corresponding improvement in therapy performance (e.g., AHI, therapy session duration, etc.). In some embodiments, the causation prediction engine 300 operates to determine a correlation coefficient (e.g., Pearson correlation formula) to measure a linear relationship between therapy performance parameter(s) and stimulation intensity over time, and from an evaluation of the correlation coefficient over time, identify possible stimulation discomfort issues. In related embodiments, the causation prediction engine 300 can operate to designate a recorded stimulation intensity value corresponding with a change in the correlation coefficient from positive to negative as a possible stimulation discomfort issue. In further related embodiments, the causation prediction engine 300 can operate to plot the determined correlation coefficient over time, and designate the recorded simulation intensity value corresponding with a slope of the plotted correlation coefficient becoming increasingly negative as a possible stimulation discomfort issue (e.g., that further increases in stimulation intensity are likely to cause larger decreases in usage).

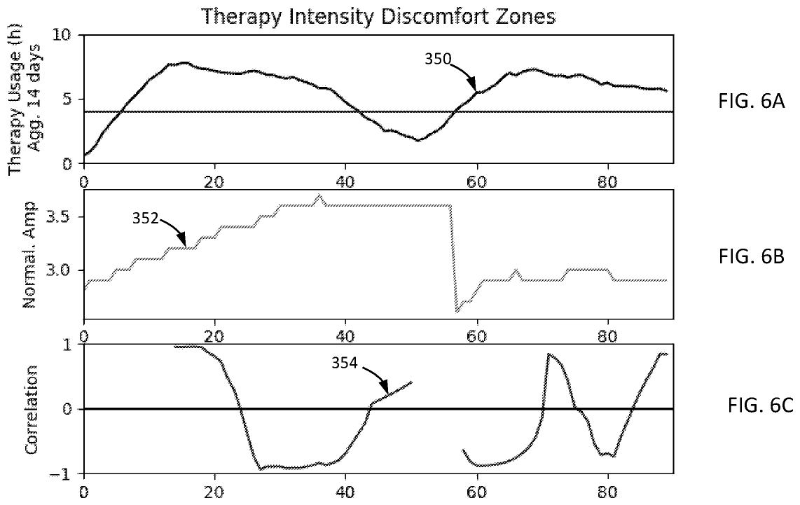

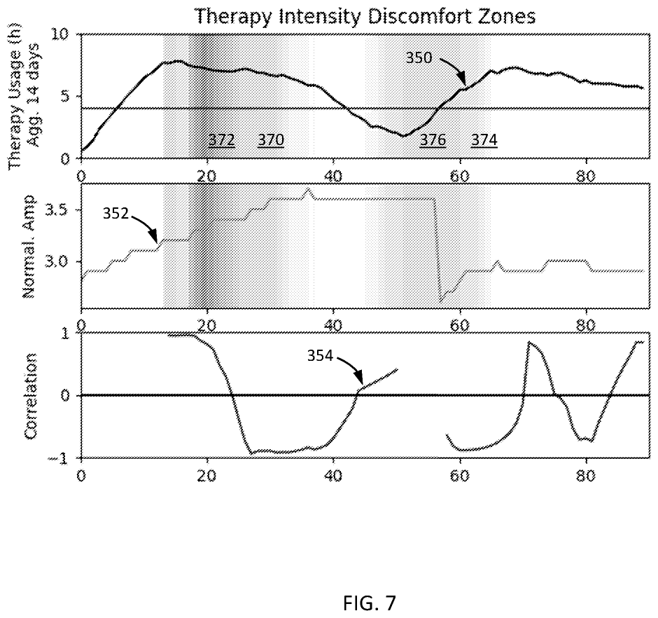

Some Therapy Discomfort Identification methods and protocols of the present disclosure are further explained with reference to the non-limiting examples of FIGS. 6A-6C. FIG. 6A is a plot 350 of daily therapy session duration (or usage) by the patient under review. FIG. 6B is a plot 352 of therapy session stimulation intensity (normalized amplitude) the same patient for the same daily therapy sessions. FIG. 6C is a plot 354 of a determined correlation coefficient between the duration and stimulation intensity values for the previous 14 days (i.e., the plot 354 begins on day 15, and the correlation coefficient is determined from the session duration and stimulation intensity data of days 1-14; day 16 of the correlation coefficient plot 354 reflects the correlation coefficient as determined from the session duration and stimulation intensity data of days 2-15; etc.). The causation prediction engine 300 can operate to review the correlation coefficient plot 354, noting stimulation intensity value(s) that otherwise correspond to the coefficient plot 354 decreasing, a slope of the coefficient plot 354 becoming increasing negative, etc., as indicative of a stimulation intensity level that is causing, or is likely to cause, discomfort, and therefore likely to cause short term and/or long term non-adherence. For example, upon the rapid increase in stimulation amplitude from day 0 to approximately day 35, and the stimulation amplitude remaining at high level (e.g., 3.6) for approximately 20 days (e.g., days 38 to day 58), a steady drop in usage hours coinciding with that stimulation amplitude pattern can be recognized by or reflected in the correlation coefficient plot 354. However, upon rapid decrease of stimulation amplitude (e.g., days 58-59), the usage duration/per day rapidly increases to quantity (e.g., 8 hours/night) above a threshold (e.g. 4 hours/night), and with future/on-going use at lower amplitudes (e.g. 3), a long term pattern develops of usage duration/per day (e.g., 6 hours/night) which is stable and efficacious. Other aggregate time frames (e.g., greater or lesser than 14 day) can also be employed. In addition, parameters other than, or in addition to, duration/usage and stimulation intensity/normalized amplitude can be utilized with the correlation analysis. For example, a similar correlation review can be performed between stimulation intensity and AHI. Also, a parameter indicative of stimulation intensity other than, or in addition to, amplitude can be reviewed, such as pulse width, etc.

In some embodiments, the causation prediction engine 300 can be programmed to (or can operate one or more protocols formatted to) generate further evaluations or characterizations of a stimulation discomfort “zone” the patient under review was experiencing at various therapy session stimulation intensities and usages based upon one or more of the correlations described above. In some non-limiting examples, the additional analyses or review can include determining the p-value (or other value indicative of statistical significance) for the correlation and/or a review of a slope of the coefficient plot 354. Various categories or rules can be applied to the obtained analysis to characterize the patient's zone or level of discomfort during previous therapy sessions. For example, some possible categories or rules can include: 1) correlation coefficient <−0.7, p-value <0.05 and slope <0, then the patient categorized as moderate discomfort zone; 2) correlation coefficient <−0.9, p-value <0.05 and slope <0, then patient categorized as substantive discomfort zone; 3) correlation coefficient <−0.7, p-value <0.05 and slope >0, then the patient categorized as moderate relief; 4) correlation coefficient <−0.9, p-value <0.05 and slope >0, then patient categorized as substantive relief zone.

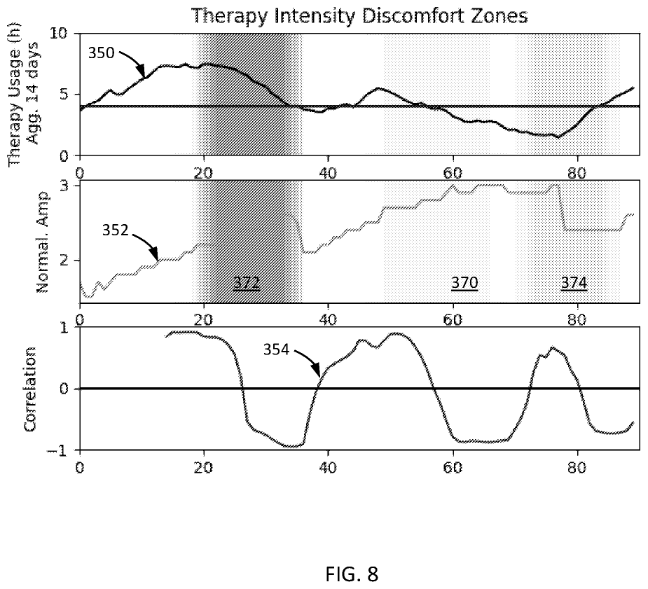

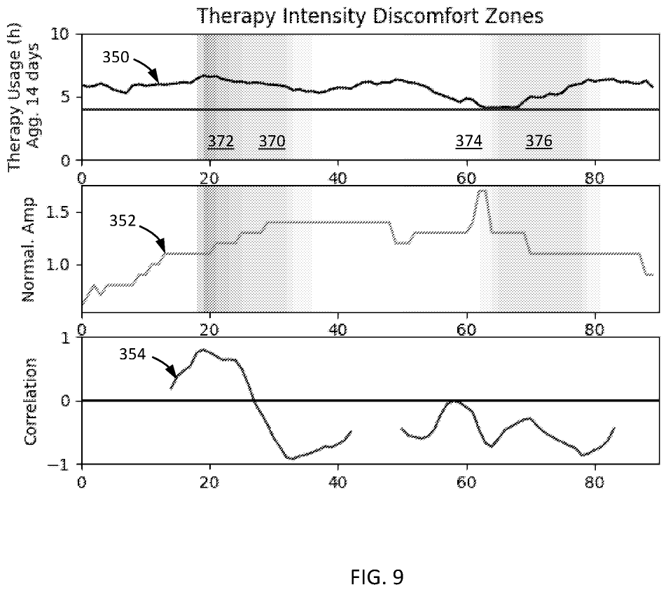

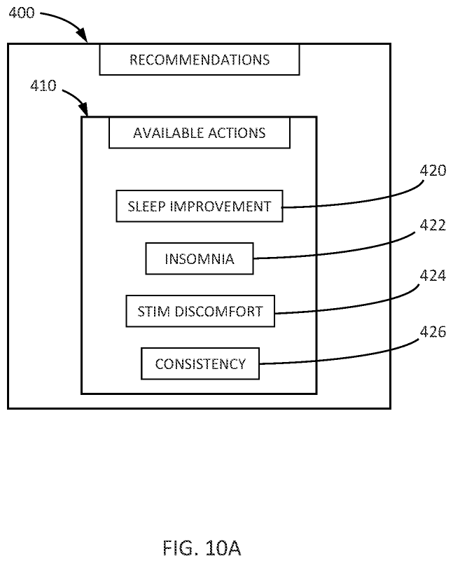

FIG. 7 provides a visual representation of some of these categories as applied or overlaid to the example patient data of FIGS. 6A-6C. In the view of FIG. 7, greyscale shading provides a visual representation of discomfort intensity. In some embodiments, the discomfort intensity can further be represented by differing colors. For example, the color yellow (and different shades thereof) can be used or added to visually represent moderate discomfort zones (e.g., general indicated at 370); the color red (and different shades thereof) can be used or added to visually represent substantive discomfort zones (generally indicated at 372); the color light blue (and different shades thereof) can be used or added to visually represent moderate relief zones (generally indicated at 374); the color dark blue (and different shades thereof) can be used or added to visually represent substantive relief zones (generally indicated at 376). Additional examples of determined stimulation intensity discomfort zones for other patient data (and optionally using the same color coding/scheme) are provided in FIGS. 8 and 9.

The discomfort zone review or analysis can be premised upon other factors and/or can generate different categories apart from those described above. The zone descriptors and/color schemes described above are but a few examples of methods and techniques in accordance with principles of the present disclosure. Other overlaying approaches, as well as other visualization techniques, can also be employed. Where the IMD 125 as arranged to the patient provides for selective stimulation at two or more different stimulation sites, a discomfort zone review or analysis can be generated on a per stimulation site basis and/or collectively for all available stimulation sites.

Returning to FIG. 1, information generated by the adherence prediction engine 200 (e.g., a predicted likelihood of patient adherence) and/or by the causation prediction engine 300 (e.g., determined likely cause(s) of patient non-adherence or potential non-adherence) can be delivered directly to one or users (e.g., one or more of the clinician devices 150). In some embodiments, the arrangement 100 can further include a recommendations engine 400. In general terms, the recommendations engine 400 is programmed to, or operates one or more protocols formatted to, identify useful actions or interventions for addressing actual or potential non-adherence of the patient under review. In some examples, the recommendations engine 400 operates on, or is managed by, the cloud resource 135.

In some embodiments, the recommendations engine 400 can have access to, or can maintain (e.g., in a memory), a number of different available actions; based upon a review of the likely or identified cause(s) of non-adherence (e.g., as generated by the causation prediction engine 300 as described above) for the patient under review, the recommendations engine 400 can select one or more of the available actions as a recommended action for the patient under review. With embodiments in which the patient under review is being treated for SBD, at least some of the available actions can be sleep-related. For example, and as reflected by FIG. 10A, some available actions 410 can include one or more sleep improvement actions 420, one or more insomnia relief actions 422, one or more stimulation discomfort actions 424, and one or more therapy consistency actions 426.

With additional reference to FIG. 10B, the sleep improvement actions 420 can include any action, activity, training, therapy, etc., that is recognized (now or in the future developed) to improve or enhance sleep quality. Some examples include sleep hygiene education 430, sleep reminders 432, sleep goal setting 434, sleep habit assessment 436, etc. The sleep hygiene education actions 430 can include providing the patient with presentations, materials (e.g., video, audio, written), and the like, tailored to explain one or more suggestions or practices that a patient can perform to encourage or preserve quality sleep. Some possible practices can include sleep environment settings, exercise, food/drink consumption, no caffeine, etc. The sleep reminders actions 432 can include a regular message or other reminder to the patient (e.g., delivered via the patient app 112 (FIG. 1)) to attempt to begin sleeping by a specified time. The sleep goal setting actions 434 can include a message or other reminder to the patient (e.g., delivered via the patient app 112) of a goal or targeted amount of sleep each night (e.g., target 8 hours of sleep per night). The sleep habit assessment actions 436 can include presenting questions to the patient that can assist in assessing a patient's normal sleep habits. A number of other sleep improvement actions 420 can also be available to the recommendations engine 400.

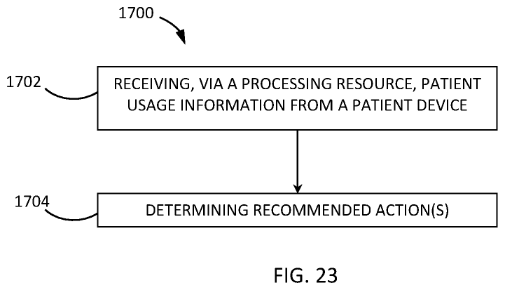

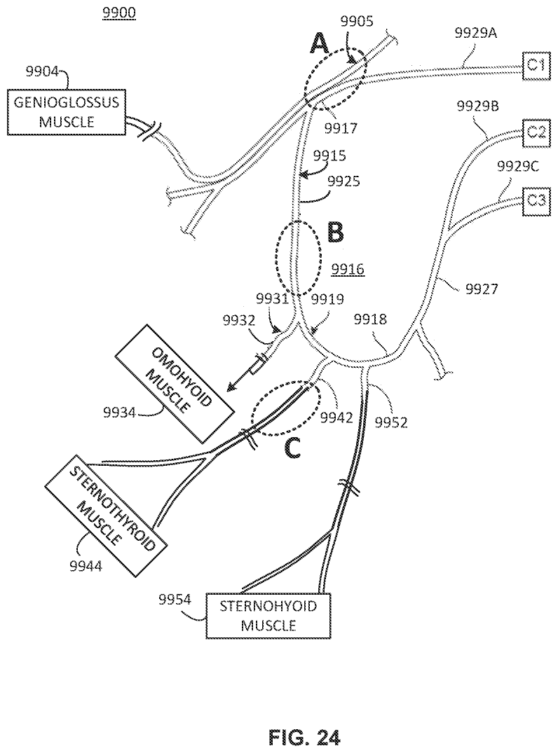

With reference to FIG. 100, the insomnia relief actions 422 can include any action, activity, training, therapy, etc., that is recognized (now or in the future developed) to alleviate or lessen insomnia. Some examples include cognitive behavior therapy (e.g., CBTI) 440, pharmaceuticals 442, insomnia assessment 444, and device programming change 446. The cognitive behavior therapy actions 440 can include enrolling the patient, or suggesting to the patient that s/he enroll and participate, in a cognitive behavior therapy treatment program or course. The pharmaceuticals actions 442 can include prescribing, or suggesting to the patient that s/he meet with a physician licensed to prescribe, drug(s) for treating insomnia. The insomnia assessment actions 444 can include presenting questions to the patient, the answers to which are useful in assessing a severity of insomnia (e.g., Insomnia Severity Index (ISI)). The device programming change actions 446 can relate to the programming or settings the IMD 125 (FIG. 1) is caused to operate under in delivering stimulation to the patient as part of a therapy session. Some of these settings can include a designated rate at which stimulation intensity increases or ramps up at the start of a therapy session, a designated delay between a “therapy on” prompt from the patient and actual delivery of stimulation, etc. With this in mind, the device programming change actions 446 for addressing insomnia can include changing (e.g., increasing) the designated rate at which stimulation intensity increases or ramps up at the start of a therapy session, changing (e.g., increasing) the designated delay following a “therapy on” prompt, etc. Other device programming change actions 446 can include switching between stimulation amplitudes of various intensities during the night (e.g., if the type of insomnia include sleep maintenance insomnia). Where the IMD 125 as arranged to the patient provides for selective stimulation at two or more different stimulation sites, the device programming change actions 446 for addressing insomnia can include recommending or changing the timing and/or intensity of stimulation delivered at each individual stimulation site during a therapy session. A number of other insomnia relief actions 422 can also be available to the recommendations engine 400.

With reference to FIG. 10D, the stimulation discomfort actions 424 can include any action or training intended to address stimulation-caused patient discomfort (actual or potential). Some examples include device programming change 450, stimulation intensity recommendation 452, discomfort assessment 454, stimulation education 456, etc.

With additional reference to FIG. 10E, the device programming change actions 450 can assume various forms based upon the particular concern(s) for the patient under review. By way of reference, some device programming permits a patient to self-increase stimulation intensity from therapy session-to-therapy session and/or during individual therapy sessions (typically within predetermined limits). With these and related embodiments, the patient can prompt an increase in stimulation intensity by a single prompt or input at a remote device (e.g., pressing a button on the patient remote control 120 (FIG. 1)); the therapy programming is such that in response to this prompt, stimulation intensity will increase at a designated rate and by designated step or amount. These and other device programming can allow a patient to increase stimulation intensity multiple times during a therapy session (typically up to a designated upper limit). With this in mind, some device programming change actions 450 can include lowering or decreasing the designated rate at which stimulation intensity increases in response to a patient prompt 460, lowering or decreasing the designated step or amount by which stimulation intensity increases in response to a patient prompt 462, limiting the number of times a patient can successfully prompt an increase in stimulation intensity during a therapy session 464, preventing patient-prompted stimulation intensity increase (e.g., lockout) 466, etc. For example, one or more of the actions 460-466 can include decreasing amplitude “steps” associated with a patient-prompted intensity increase by reducing pulse width or enabling smaller amplitude steps (e.g., changing designated stimulation increase amount from 0.1V to 0.05V); locking out the ability of the patient to increase stimulation intensity for X number of days.

By way of further reference, some device programming permits a patient to temporarily pause delivery of stimulation during a therapy session by a prompt or input at a remote device (e.g., pressing a button on the patient remote control 120); in response to this prompt, stimulation will stop for a designated length of time (or “pause duration”), then return to a designated stimulation intensity level (or “after-pause intensity”, typically the intensity immediately prior to the pause request) at a designated rate (or “after-pause ramp rate”). With this in mind, some device programming change actions 450 can include increasing the designated pause duration 468 (e.g., for patients determined to have engaged in pause-chaining by prompting a number of consecutive “pause” commands), decreasing the designated after-pause ramp rate 470, decreasing the designated after-pause intensity (e.g., a stimulation intensity lower than that being delivered prior to the pause prompt) 472, etc.

A number of other device programming change actions 450 can also be available to the recommendations engine 400 for addressing stimulation discomfort. For example, some device programming establishes a designated rate at which stimulation intensity increases (from zero or no stimulation) at the start of a therapy session. With this in mind, some device programming change actions 450 can include changing (e.g., lowering or decreasing) the designated rate at which stimulation intensity increases at the start of a therapy session 474 (e.g., following a “therapy on” command). In other examples, some device programming establishes a designated rate at which stimulation intensity decreases (to zero or no stimulation) at the end of a therapy session. With this in mind, some device programming change actions 450 can include changing (e.g., raising or increasing) the designated rate at which stimulation intensity decreases at the end of a therapy session 476 (e.g., following a “therapy off” command). In other examples, some device programming facilitates the detection and determination of whether the patient is awake or asleep; other operations (e.g., delivering stimulation, ending stimulation, etc.) are performed as a function of the determination. With these and related embodiments, the device programming affords an ability to alter a designated sensitivity of the sleep/awake determination protocols. With this in mind, some device programming change actions 450 can include changing (e.g., increasing) the designated sensitivity of sleep/awake determination 478.

Where the IMD 125 as arranged to the patient provides for selective stimulation at two or more different stimulation sites, the device programming change actions 450 for addressing discomfort can include stimulation site changes 479, for example, recommending or changing the timing and/or intensity of stimulation delivered at each individual stimulation site during a therapy session. By way of non-limiting example, where the IMD 125 is arranged to selectively stimulate a hypoglossal nerve and an IHM-innervating nerve of the patient, the programming change(s) can include switching from hypoglossal nerve stimulation to IHM-innervating nerve stimulation at the end of the night under circumstances where the patient is determined to have growing hypoglossal nerve stimulation intolerance over the night (for many patients, IHM-innervating nerve stimulation is more comfortable/tolerable than hypoglossal nerve stimulation). Alternatively or additionally, for a patient found to frequently pause therapy at the beginning of the night, the programming change(s) can include initially stimulating only the IHM-innervating nerve at the start of a therapy session, then add in hypoglossal nerve stimulation as the patient goes into deeper sleep. Alternatively or additionally, programmed stimulation intensity at a first one of the available stimulation sites can be decreased while programmed stimulation intensity at a second one of the available stimulation sites can be increased. A number of other programming changes can be implemented relative to timing and/or intensity of stimulation delivered to each stimulation site based upon determined patient discomfort.

Returning to FIG. 10D, the stimulation intensity recommendation actions 452 can assume various forms, and generally entail providing the patient with information relating to stimulation intensity levels. For example, a recommended stimulation intensity value or setting can be provided to the patient with or without additional supporting information. Under circumstances, for example, where it is determined that the patient has been increasing stimulation intensity beyond a designated level with minimal or no therapeutic benefit (e.g., AHI is found to be satisfactory at a designated intensity level, but the patient is continuing to prompt increases above the designated intensity level, or symptoms are satisfactorily improved but the patient is continuing to increase stimulation intensity and therapy usage is decreasing), a recommendation can be provided to the patient to use or not exceed the designated level. Additional supporting information can include a summary of previous usage data/stimulation intensities and resulting therapeutic effect. Other recommendations and/or supporting information can include results of the discomfort zone review or analysis described above.

A number of other stimulation intensity recommendation actions 452 can also be available to the recommendations engine 400 for addressing stimulation discomfort. For example, where the IMD 125 as arranged to the patient provides for selective stimulation at two or more different stimulation sites and the patient is provided with the ability to select or alter the site(s) receiving stimulation during a therapy session, the simulation intensity recommendation actions 452 can include recommending that the patient select or choose the stimulation site found to be more comfortable/tolerable. In other situations, recommendations can include adding a new stimulation site, upgrading the IPG 125, etc.

The discomfort assessment actions 454 can include presenting questions to the patient, the answers to which can assist in assessing a patient's stimulation-related discomfort.

The stimulation education actions 456 can include can include providing the patient with presentations, materials (e.g., video, audio, written), and the like, that include suggestions to the patient of ways to alleviate stimulation therapy discomfort, follow a therapy plan, etc.

A number of other stimulation discomfort actions 424 can also be available to the recommendations engine 400.

Returning to FIG. 10A and with additional reference to FIG. 10F, the therapy consistency actions 426 can include any action or training intended to encourage a patient to more consistently adhere to a treatment plan/engage in beneficial therapy sessions. Some examples include therapy start time goal reminder 480, therapy end time goal reminder 482, therapy duration goal reminder 484, therapy pause goal reminder 486, unintended therapy off command goal reminder 488, halted therapy goal reminder 490, missed therapy session goal reminder 492, stimulation intensity goal reminder 494, device programming change 496, stimulation education 498, and cognitive behavior therapy 500.

The therapy start time goal reminder actions 480 can include providing the patient with information relating to a recommended time of day (or night) for starting a therapy session. For example, a recommendation or reminder of start time for daily/nightly therapy sessions can be provided to the patient with or without additional supporting information. The recommended daily/nightly therapy session start time can be derived from the therapy plan assigned to the patient. Additional supporting information can include a summary of previous usage data relevant to therapy session start time, for example a parameter indicative of the number of previous days over a designated time frame (e.g., two weeks, one month, etc.) in which a therapy session start time deviated from the recommended start time by more than a predetermined length of time (e.g., 30 minutes). Further supporting information could be derived from environmental factors that are supportive of healthy circadian rhythm, such as sunset/sunrise times, latitude, patient demographics, etc. With these and related embodiments, optimal times for sleeping can be premised upon a derived or predicted circadian rhythm of the patient under review.

The therapy end time goal reminder actions 482 can include providing the patient with information relating to a time of day for ending a therapy session. For example, a recommendation or reminder of end time for daily/nightly therapy sessions can be provided to the patient with or without additional supporting information. The recommended daily/nightly therapy session end time can be derived from the therapy plan assigned to the patient. Additional supporting information can include a summary of previous usage data relevant to therapy session end time, for example a parameter indicative of the number of previous days over a designated time frame (e.g., two weeks, one month, etc.) in which a therapy session end time deviated from the recommended end time by more than a predetermined length of time (e.g., 30 minutes), etc.

The therapy duration goal reminder actions 484 can include providing the patient with information relating to therapy session duration. For example, a recommendation or reminder of therapy session duration can be provided to the patient with or without additional supporting information. The recommended therapy session duration can be derived from the therapy plan assigned to the patient. Additional supporting information can include a summary of previous usage data relevant to therapy session duration, for example a parameter indicative of the average therapy session duration over a designated time frame (e.g., two weeks, one month, etc.), the number of therapy sessions over a designated time frame (e.g., two weeks, one month, etc.) having a duration less than a predetermined length of time (e.g., 4 hours), etc.

The therapy pause goal reminder actions 486 can include providing the patient with information relating to therapy session pauses. For example, a recommendation or reminder to limit the number therapy session pauses can be provided to the patient with or without additional supporting information. Additional supporting information can include a summary of previous usage data relevant to therapy session pauses, for example a parameter indicative of the average number of therapy session pauses over a designated time frame (e.g., two weeks, one month, etc.), the number of therapy sessions over a designated time frame (e.g., two weeks, one month, etc.) having a number of pauses exceeding a predetermined number (e.g., 2 pauses), etc.

The unintended therapy off command goal reminder actions 488 can include providing the patient with information relating to “therapy off” commands given during a therapy session. By way of background, in some instances, a patient intending to effect a “therapy pause” command during a therapy session may mistakenly instead prompt a “therapy off” command. Following this unintended “therapy off” command, the patient then re-starts the therapy session at a later point in time, with the delay between the “therapy off” and subsequent “therapy on” commands being greater than the duration of a “pause” command. With this mind, in some examples, a recommendation or reminder to limit the number “therapy off” commands during a therapy session can be provided to the patient with or without additional supporting information. Additional supporting information can include a summary of previous usage data relevant to “therapy off” commands during therapy sessions, for example a parameter indicative of the average number of “therapy off” commands prompted by the patient during therapy sessions over a designated time frame (e.g., two weeks, one month, etc.), the number of therapy sessions over a designated time frame (e.g., two weeks, one month, etc.) having a number of “therapy off” commands exceeding a predetermined number (e.g., one “therapy off” command), etc.

The halted therapy goal reminder actions 490 can include providing the patient with information relating to halted therapy sessions. By way of background, a halted therapy session can be a therapy session lasting less than a predetermined length of time (e.g., 1 hour). With this in mind, in some examples, a recommendation or reminder to avoid halted therapy sessions can be provided to the patient with or without additional supporting information. Additional supporting information can include a summary of previous usage data relevant to halted therapy session, for example a parameter indicative of the average number of halted therapy sessions over a designated time frame (e.g., two weeks, one month, etc.), etc.

The missed therapy session goal reminder actions 492 can include providing the patient with information relating to missed therapy sessions. By way of background, many treatment plans recommend that the patient perform each therapy session on a regular basis; for example, some SDB treatment plans recommend that the patient perform stimulation therapy every evening when sleeping. In some examples, a recommendation or reminder to avoid missed therapy sessions can be provided to the patient with or without additional supporting information. Additional supporting information can include a summary of previous usage data relevant to missed therapy sessions, for example a parameter indicative of the number of missed therapy sessions over a designated time frame (e.g., two weeks, one month, etc.), etc.

The stimulation intensity goal reminder actions 494 can include providing the patient with information relating to recommended therapy session stimulation intensities. As a point of reference, some device programming permits a patient to self-decrease (or self-increase) stimulation intensity from therapy session-to-therapy session and/or during individual therapy sessions (typically within predetermined limits). Further, some treatment plans recommend, or present as a goal, a minimum stimulation intensity level for therapy sessions; with some treatment plans, the recommended minimum stimulation intensity level may increase over time (e.g., during initial stages of a therapy program, a recommended minimum stimulation intensity level may be relatively low as the patient is becoming accustomed to the stimulation therapy; later, the recommended minimum stimulation intensity level is increased to enhance the therapeutic effect). With these and related embodiments, the patient may self-select a stimulation intensity level that is less than the recommended stimulation intensity level for a particular therapy session. With this in mind, some stimulation intensity goal reminder actions 494 can include a recommendation or reminder to increase the stimulation intensity selected by the patient for future therapy sessions; this recommendation or reminder can be provided to the patient with or without additional supporting information. Additional supporting information can include a summary of previous usage data relevant to stimulation intensity, for example a parameter indicative of the stimulation intensity used by the patient over a designated time frame (e.g., two weeks, one month, etc.), a comparison of the patient's recently selected stimulation intensities with other, similarly-situated patients, etc.

The device programming change actions 496 can assume various forms based upon the particular concern(s) for the patient under review. As a point of reference, some device programming permits a patient to temporarily pause delivery of stimulation during a therapy session by a prompt or input at a remote device (e.g., pressing a button on the patient remote control 120 (FIG. 1)); in response to this prompt, stimulation will stop for a designated length of time (or “pause duration”), then return to a designated stimulation intensity level (or “after-pause intensity”, typically the intensity immediately prior to the pause request) at a designated rate (or “after-pause ramp rate”). With this in mind, some device programming change actions 496 can include increasing the designated pause duration, decreasing the designated after-pause ramp rate, etc., (for example to address a patient found to be prompting an excessive number of “pause” commands during therapy sessions). For patients determined to have engaged in pause-chaining (i.e., prompting a number of consecutive “pause” commands), the device programming change actions 496 can include increasing the programmed pause duration.

As further examples, some device programming automatically initiates a therapy session at a designated time of day, automatically ends a therapy session at a designated time of day, and/or establishes a designated duration for therapy sessions. With this in mind, some device programming change actions 496 can include changing one or more of the designated therapy session start time, the designated therapy session end time, the designated therapy session duration, etc., (for example to address a patient found to have irregular sleep times such as weekend sleep times vs. weekday sleep times).

A number of other device programming change actions 496 can also be available to the recommendations engine 400 for addressing therapy consistency.

The stimulation education actions 498 can include can include providing the patient with presentations, materials (e.g., video, audio, written), and the like, that include suggestions or instructions for the patient regarding best practices for consistent stimulation therapy.

The cognitive behavior therapy actions 500 can include enrolling the patient, or suggesting to the patient that s/he enroll, in a cognitive behavior therapy treatment program tailored to assist a patient in achieving more consistent usage of stimulation therapy.

A number of other therapy consistency actions 426 can also be available to the recommendations engine 400.

Returning to FIGS. 1 and 10A, regardless of the actions or interventions available for consideration, the recommendations engine 400 is programmed to, or operates one or more protocols formatted to, select a targeted action(s) for the patient under review based upon assessed or determined concern(s), for example as generated by the causation prediction engine 300. In some embodiments, the recommendations engine 400 can reference predetermined rules or standards to select a targeted action(s) in response to an identified or assessed concern. In some embodiments, the recommendations engine 400 has access to usage information, assessed concerns, and observed success of actions/interventions (or models of such information) by previous patient (e.g., via the database 202); with these and other embodiments, the recommendations engine 400 can operate to compare the assessed or determined concern(s) and collected information (e.g., previous usage data) for the patient under review with other, similarly-situated patients or groups of patients to select a target action(s) for the patient under review, for example based on group observations, cluster analysis, etc. In some examples, the recommendations engine 400 can consider the success of previous targeted actions with the patient under review when selecting or evaluating future actions for the patient.

In some embodiments, where two or more recommended actions are selected, or otherwise deemed beneficial, for the patient under review, the recommendations engine 400 is programmed to, or operates one or more protocols formatted to, prioritize the selected targeted actions. For example, the recommendations engine 400 can prioritize targeted actions based upon determined severity of the assessed concerns (e.g., for a patient determined as having substantial sleep hygiene issues and periodic therapy consistency concerns, the recommendations engine 400 can prioritize one or more of the sleep improvement actions 420 over the therapy consistency actions 426). In other embodiments, the recommendations engine 400 can utilize group observations, cluster analysis, etc., as described above to prioritize selected targeted actions for the patient under review.

In some embodiments, the recommendations engine 400 can prioritize selected targeted actions based upon a review of the collected usage data for the patient under review. For example, the recommendations engine 400 can be programmed to, or can operate one or more protocols formatted to, review specified usage information deemed relevant to one or more behaviors indicative of therapy consistence concerns, and derive prioritize targeted actions based upon the usage behaviors found to be most erratic. As a point of reference, it has been observed that consistency in daily/nightly therapy sessions can be a primary attribute of long term therapy success. With this in mind, and by way of non-limiting example, some usage behaviors indicative of consistent therapy usage can include therapy start time, therapy end time, therapy duration, therapy pause, unintended therapy off command, halted therapy, and missed therapy session as described above. These, and optionally other, behaviors, are implicated by patient data, for example by the timestamped patient remote data discussed above. The recommendations engine 400 optionally can review the collected patient remote data, and identify those behaviors with the largest inconsistencies as points of focus or priority. The review can be based upon a statistical analysis of the collected usage data. For example, a standard deviation can be computed for the behaviors, normalized by standard scaler. A 0 to 1 normalization can then be performed to derive a percentage “score” for each behavior as based on standard deviations as compared to one another. The behaviors determined to have the highest score can then be designated as action focus points or priorities. The chart of FIG. 11 is an example of possible analyses/protocols operated by the recommendations engine 400 based upon hypothetical collected patient remote data.

In some embodiments, the recommendations engine 400 can be programmed to, or operate one or more protocols formatted to, generate one or more displays that highlight prioritized targeted actions. For example, continuing with the above embodiments in which the patient behaviors are scaled and normalized relative to one another, the resultant scores can be presented in a report that highlights points of focus. FIG. 12 is one example of a representative report 600 generated from a review of collected patient remote data 602. The report 600 highlights patient behaviors/actions determined to be most erratic, thus providing an easy-to-understand prioritization of recommended actions for improving therapy usage as a form of personalized therapy (e.g., specific to the individual patient under review).

The analysis of FIG. 11 and the report of FIG. 12 are but two non-limiting examples of prioritization operations that can be performed by the recommendations engine 400. A wide variety of other techniques can be employed to designate or highlight which, of multiple available actions, selected targeted actions can be prioritized for a patient under review.

Returning to FIG. 1, information generated by one or more or all of the adherence prediction engine 200, the causation prediction engine 300, and the recommendations engine 400 can be delivered or reported to one or more of a treating clinician or clinician entity (e.g., via one of the clinician devices 150) and/or the patient (e.g., via the patient app 112) as a form of personalized therapy (e.g., specific to the individual patient under review), for example via operations of a reporting and implementation engine 700. In some examples, information (optionally along with other personalized therapy) as delivered to the treating clinician or clinician entity (e.g., at the corresponding clinician device 150) by the reporting and implementations engine 700 can include the determined adherence prediction (e.g., via the adherence prediction engine 200), the determined likely causes (e.g., via the causation prediction engine 300), the selected targeted actions (e.g., via the recommendations engine 400), and optional supporting information as described below. From the so-provided information, the treating clinician can make further determinations, and can choose whether or not the selected targeted actions will be implemented, for example via the portal 155, to further enhance personalized therapy. In some examples, the reporting and recommendations engine 700 operates on, or is managed by, the cloud resource 135.

FIG. 13 is one non-limiting example of a clinician report 710 that can be generated by the reporting and implementations engine 700, and delivered to a treating clinician (for example at the corresponding portal 155 (FIG. 1)) for a particular patient. With reference between FIGS. 1 and 13, the clinician report 710 is an example of personalized therapy and provides a determined adherence prediction 720, determined likely causes of non-adherence 722, and recommended actions 724. The clinician report 710 can optionally include additional information such as usage summary data 726 (e.g., number of nights used of a total number of nights, number of nights used over 4 hours of a total number of nights) and a display of actual usage 728 on a night-by-night basis. In some embodiments, the clinician report 710 can provide a reviewing clinician with the ability to access or review additional data supporting one or more of the identified likely causes of non-adherence 722, for example via a provided link(s) 730. In some embodiments, the clinician report 710 can provide a reviewing clinician with a listing of one or more additional, potential causes of non-adherence 740. The potential causes of non-adherence 740 can be determined or generated by the causation prediction engine 300, for example as being potentially relevant to non-adherence, but deemed as being less likely as compared to the determined causes of non-adherence 722. With these and related embodiments, and under circumstances where the treating clinician views one (or more) of the additional, potential causes of non-adherence 740 as being a likely cause, the treating clinician can be provided with the ability (e.g., via the portal 155) to select or identify the so-viewed potential cause; this clinician identification can be delivered, via the reporting and implementation engine 700, to the causation prediction engine 300 for further consideration (e.g., in some embodiments, the protocol(s) operated by the causation prediction engine 300 can account for or include clinician-provided information). Where the clinician-provided information changes the likely causes of non-adherence as determined by the causation prediction engine 300 and/or the recommended actions as determined by the recommendations engine 400, a new clinician report can be generated.

The clinician report 710 can further provide a treating clinician to select or “order” one or more or all of the recommended actions 724. In some embodiments, the reporting and implementation engine 700 can operate to automatically perform or effect one or more of the selected recommended actions 724. For example, where a selected recommended action is of a type that can be implemented via the patient app 112, the reporting and implementation engine 700 operates to prompt performance of the selected recommended action at the patient's app (e.g., by the clinician selecting or clicking, at the portal 155, the “Send to app” icon 750). Other types of recommended actions may need to be performed by the treating clinician (e.g., some device programming actions as described above, addition of another stimulation site, etc.). In yet other embodiments, a recommended action could be automatically effected or implemented by the reporting and implementation engine 700 without clinician approval, for example where the patient under review has been receiving/performing stimulation therapy for an extended length of time and the determined likely cause of non-adherence/recommended action is deemed to meet predetermined criteria.

Another non-limiting example of information or displays that can be provided in a clinician report 760 is shown in FIG. 14. The clinician report 760 is another example of personalized therapy and can include a visual representation or summary of patient usage behavior 770 and/or a visual representation or summary of stimulation discomfort information 772. The summary of patient usage behavior 770 can assume a wide variety of forms, and in some examples can be, or can be akin to, a display that highlights patient behaviors/actions determined to be most erratic, thus providing an easy-to-understand prioritization of recommended actions for improving therapy usage as described above. The summary of stimulation discomfort information 772 can also assume a wide variety of forms, and in some embodiments can be, or can be akin to, a color-coded display of determined stimulation intensity discomfort zones as described above. In yet other embodiments, the clinician report can provide a side-by-side comparison or illustration of various parameters of the patient along with adherence status (e.g., AHI, Epworth Sleep Scale (ESS), and adherence status), for example as a 3-bar plot. Other useful clinician report examples are provided in U.S. application Ser. No. 18/196,680 filed May 12, 2023 and entitled “Clinician User Interface” the entire teachings of which are incorporated herein by reference.

Returning to FIG. 1, in some example arrangements, a clinician is apprised of and/or can manage multiple different patients, each using a similar stimulation treatment therapy, via the clinician portal 155. With these and related embodiments, the clinician portal 155 can periodically provide summary-type lists or reports (e.g., generated at or by the cloud resource 135) for all similarly-situated patients (e.g., patients being treated for SDB with stimulation therapy provided by an IMD) under the care of the clinician or other user of the clinician device 150. With this in mind, in some examples, the reporting and implementations engine 700 is programmed to, or operates one or more protocols formatted to, generate a prioritized listing of the similarly-situated patients under the care of the clinician that highlights those patients requiring more immediate attention for actual or predicted non-adherence. The so-generated prioritized report or listing can be made available or viewed at the clinician portal 155. Prioritization can be based upon various factors, such as, but not limited to, predicted adherence issues, clustering, etc., and can highlight patients deemed to need more immediate attention or otherwise as being comparatively more problematic in various manner. For example, patients requiring more immediate attention can be placed at the beginning or top of the listing of all patient. One non-limiting example of a prioritized patient list report 780 is provided in FIG. 15, and prioritizes patients by various factors 782 (e.g., suspected sleep issues, suspected stimulation discomfort concerns, and days without usage). For each patient listed, the report 780 can include an indication 784 of a severity of the particular concern (e.g., number of hours the particular concern occurred in the corresponding patient), along with a link 786 to more detailed information.

In other examples of priority patient list reports in accordance with principles of the present disclosure, all patients under the care of a clinician for treatment of a similar malady (e.g., SDB) are first classified into groups, and then presented to the clinician by grouping. For example, all patients of the clinician being treated for the same or similar malady can be classified into one of 3 or 4 (or more) groups based on current adherence or the like. In some examples, a patient's determined and/or predicted adherence can be assigned a score or classification in accordance with a basic or general scale of 1-3, 1-4, etc. (e.g., akin to the New York Heart Association (NYHA) Functional Classification), and all of the clinician's patient with the same adherence score or classification are grouped together for convenient review by the clinician (e.g., on the clinician portal 155). The categories or classifications can have readily-understood meanings, for example: Class 1—high adherence, good AHI and good ESS; Class 2—good AHI, but either low adherence or bad ESS or both; Class 3—bad AHI. Another non-limiting example of classifications or categories is: Class 1/high adherence to treatment plan (e.g., at least 90% adherent; Class 2/good adherence to treatment plant (e.g., 70-89% adherent); Class 3/some adherence to treatment plan (e.g., 50-69% adherent); Class 4/poor adherence to treatment plan (e.g., less than 50% adherent). One non-limiting example of a categorized display or report 790 as provided to the clinician (e.g., as a graphical user interface) is shown in FIG. 16. This arrangement provides the clinician with the ability to more quickly identify and/or prioritize patients needing attention. Further, groups of patients between clinicians can be more readily compared based upon a similar score or classification. In some embodiments, the score or classification assigned to a particular patient can dictate a particular or specific intervention. Moreover, a patient's score or classification can be tracked to measure whether a particular intervention resulted in an improved score.

Returning to FIG. 1, the reporting and implementation engine 700 can be programmed to generate or report information to the patient (e.g., via the patient app 112) in a wide variety of forms. By interacting with the patient app 112, the patient is more engaged in the treatment plan and thus more likely to adhere to a particular treatment plan. One non-limiting example of a patient report 800 is shown in FIG. 17. The patient report 800 is another example of personalized therapy and can provide recommended actions 810 (for example, as determined by the recommendation engine 400 as described above). Where two (or more) recommended action 810 are presented, the patient report 800 can highlight (e.g., color, font, type size, etc.) a particular one of the recommended actions 810 to the patient. The patient report 800 can optionally include additional information such as usage summary data 820.

Returning to FIG. 1, the reporting and implementation engine 700 can be programmed to provide or implement recommended action(s) to the patient on an interactive basis in some embodiments in conjunction with personalized therapy for the patient. Several examples of possible implementations via the patient app 112 are provided in FIGS. 18A-18D (that otherwise depict the display screen of a patient handheld device operating the patient app).

The example display 900 of FIG. 18A (otherwise presented on the patient device 110) reflects one possible implementation of a recommended action for addressing determined or predicted non-adherence due to sleep issues, and in particular a patient deemed to sleep later than expected. The display 900 includes or provides several recommended actions, including a reminder goal 902 (e.g., in the form of a friendly “tip” to the patient), an education action 904 (e.g., a link to a relevant educational video), and a sleep reminder action 906 (e.g., a link to a screen at which the patient can establish sleep reminder settings).

The example display 910 of FIG. 18B reflects one possible implementation of a recommended action for addressing determined or predicted non-adherence due to insomnia. The display 910 includes or provides several recommended actions, including a recommendation action to participate in a cognitive behavior therapy (CPTI) course 912, and an education action 914 (e.g., a link to a cognitive behavior therapy CBTI course).

The example display 920 of FIG. 18C reflects one possible implementation of a recommended action for addressing determined or predicted non-adherence due to stimulation discomfort issues. The display 920 includes or provides several recommended actions, including a discomfort assessment action 922 (e.g., in the form of a question to the patient), and stimulation education actions 924 (e.g., a friendly “tip” to the patient and a link to a relevant educational video).

The example display 930 of FIG. 18D reflects one possible implementation of a recommended action for addressing determined or predicted non-adherence due to therapy consistency issues, and in particular a patient deemed to be self-selecting a lower than recommended stimulation intensity. The display 930 includes or provides several actions, including a stimulation intensity goal reminder action 932 (e.g., in the form of a status of the status of the patient's usage and a comparison with other patients), a stimulation education action 934 (e.g., a link to a relevant educational video), and a cognitive behavior therapy action 936 (e.g., a link to a cognitive behavior therapy (CBTI) course).

With any of the recommended action(s) generated displays as provided to the patient (e.g., via the patient app 112 (FIG. 1)), text can be included that explains to the patient that other, similarly-situated patients had found the action(s) being recommended to be useful.

FIG. 19 is a block diagram schematically representing an example control portion 1050. In some examples, the control portion 1050 provides one example implementation of a control portion forming a part of, implementing, and/or generally managing the example arrangements, the implantable medical devices (IMDs, e.g. IPG), cloud resources, clinician devices, patient remotes, patient apps, programmers, user interfaces, control portion, instructions, workflows, engines, functions, parameters, and/or methods, as described throughout examples of the present disclosure in association with FIGS. 1-18D. In some examples, all or some of the features of the adherence prediction engine 200, the causation prediction engine 300, the recommendations engine 500, and/or the reporting and implementations engine 700 may be implemented via, and/or as part of, control portion 1050. In some examples, the control portion 1050 includes a controller 1052 and a memory 1060. In general terms, the controller 1052 of the control portion 1050 comprises at least one processor 1054 and associated memories. The controller 1052 is electrically couplable to, and in communication with, the memory 1060 to generate control signals to direct operation of at least some of the example arrangements, IMDs, clinician devices, cloud resources, patient remotes, patient apps, user interfaces, control portion, instructions, workflows, engines, functions, parameters, and/or methods, as described throughout examples of the present disclosure. In some examples, these generated control signals include, but are not limited to, employing instructions 1061 and/or information 1062 stored in the memory 1060 to at least direct and manage sleep disordered breathing (SDB) care (e.g. sensing, stimulation, related patient management, adherence prediction, non-adherence causes, recommendations, etc.) in the manner described in at least some examples of the present disclosure. In some instances, the controller 1052 or the control portion 1050 may sometimes be referred to as being programmed to perform the above-identified actions, functions, etc.

In response to or based upon commands received via a user interface (e.g. user interface 1070 in FIG. 20 or example user interfaces throughout FIGS. 1-18D) and/or via machine readable instructions, the controller 1052 generates control signals as described above in accordance with at least some of the examples of the present disclosure. In some examples, the controller 1052 is embodied in a general purpose computing device while in some examples, the controller 1052 is incorporated into or associated with at least some of the example arrangements, IMDs, clinician devices, cloud resources, patient remotes, patient apps, user interfaces, control portion, instructions, workflows, engines, functions, parameters, and/or methods, etc. as described throughout examples of the present disclosure.

For purposes of this application, in reference to the controller 1052, the term “processor” shall mean a presently developed or future developed processor (or processing resources) that executes machine readable instructions contained in a memory or that includes circuitry to perform computations. In some examples, execution of the machine readable instructions, such as those provided via the memory 1060 of the control portion 1050 cause the processor to perform the above-identified actions, such as operating controller 1052 to implement one or more of adherence prediction, causation prediction, recommendations, reporting and implementation, and patient care via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by the memory 1060. The machine readable instructions may include a sequence of instructions, a processor-executable machine learning model, or the like. In some examples, the memory 1060 comprises a computer readable tangible medium providing non-volatile storage of the machine readable instructions executable by a process of the controller 1052. In some examples, the computer readable tangible medium may sometimes be referred to as, and/or comprise at least a portion of, a computer program product. In other examples, hard wired circuitry may be used in place of or in combination with machine readable instructions to implement the functions described. For example, the controller 1052 may be embodied as part of at least one application-specific integrated circuit (ASIC), at least one field-programmable gate array (FPGA), and/or the like. In at least some examples, the controller 1052 is not limited to any specific combination of hardware circuitry and machine readable instructions, nor limited to any particular source for the machine readable instructions executed by the controller 1052.

In some examples, the control portion 1050 may be entirely implemented within or by a stand-alone device.

In some examples, the control portion 1050 may be partially implemented in one of the example arrangements, cloud resources, clinician devices, patient remotes, patient apps, IMDs, etc. and partially implemented in a computing resource separate from, and independent of, the example arrangements, cloud resources, clinician devices, patient remotes, patient apps, IMDs, etc. but in communication with such example arrangements, etc. For instance, in some examples the control portion 1050 may be implemented via a server accessible via the cloud and/or other network pathways. In some examples, the control portion 1050 may be distributed or apportioned among multiple devices or resources such as among a server, an example arrangement, and/or a user interface.

In some examples, the control portion 1050 includes, and/or is in communication with, a user interface 1070 as shown in FIG. 20. In some examples, at least some portions or aspects of the user interface 1070 are provided via a graphical user interface (GUI), and may comprise a display 1074 and input 1072. In some examples, the user interface 1070 comprises a user interface or other display that provides for the simultaneous display, activation, and/or operation of at least some of the example arrangements, cloud resources, clinician devices, patient remotes, patient apps, IMDs, control portion, workflows, instructions, engines, functions, parameters, and/or methods, etc., as described in association with FIGS. 1-18D. For instance, the various user interfaces described in association with FIGS. 1-18D may each provide an example implementation of user interface 1070.

FIG. 21 is a flow diagram schematically representing an example method 1500 of patient management. In some examples, method 1500 may be implemented via at least some the example arrangements, patient remote, patient app, processing resource, clinician portals, engines, parameters, functions, user interfaces, control portions, etc., as described in association with at least FIGS. 1-20. In some examples, method 1500 may be implemented via at least some elements, arrangements, patient remote, patient app, processing resource, clinician portals, engines, parameters, functions, user interfaces, control portions, etc. other than those described in association with at least FIGS. 1-20.

As shown at 1502 in FIG. 21, in some examples method 1500 comprises receiving, via a processing resource, usage information of a patient under review from a patient app or other resource. In some such examples, the method may comprise the patient app receiving or generating the usage information from a patient remote control. The patient remote control may receive at least some of the patient information from an implantable medical device and/or may produce some of the patient information from the patient remote control tracking and storing patient usage of the patient remote control.

In some examples, the method 1500 may further comprise the receiving externally monitored patient information comprising receiving sensed physiologic information including at least one of: cardiac information; respiratory information; chest motion; oxygen desaturation information; peripheral arterial information; blood pressure; body position; and acoustic information including snoring.

In some examples, the method 1500 may further comprise the receiving the externally monitored patient information comprising receiving determined information including at least one of: sleep disorder breathing (SDB)-related index information; sleep information; and cardiac information.

As shown at 1504 in FIG. 21, in some examples method 1500 comprises determining or predicting a likelihood that the patient under review will adhere to a treatment plan based upon a review or comparison of the usage information with previous patient usage information or a model thereof. In some embodiments, the determined or predicted likelihood of patient adherence (or non-adherence) can be displayed to a clinician. Alternatively or in addition, the determined or predicted likelihood of patient adherence (or non-adherence) and/or information utilized to arrive at the determined or predicted likelihood of patient adherence can initiate other methods of the present disclosure.

FIG. 22 is a flow diagram schematically representing an example method 1600 of patient management. In some examples, method 1600 may be implemented via at least some the example arrangements, patient remote, patient app, processing resource, clinician portals, engines, parameters, functions, user interfaces, control portions, etc., as described in association with at least FIGS. 1-21. In some examples, method 1600 may be implemented via at least some elements, arrangements, patient remote, patient app, processing resource, clinician portals, engines, parameters, functions, user interfaces, control portions, etc. other than those described in association with at least FIGS. 1-21.

As shown at 1602 in FIG. 22, in some examples method 1600 comprises receiving, via a processing resource, usage information of a patient under review from a patient app or other resource. In some such examples, the method may comprise the patient app receiving or generating the usage information from a patient remote control. The patient remote control may receive at least some of the patient information from an implantable medical device and/or may produce some of the patient information from the patient remote control tracking and storing patient usage of the patient remote control.

In some examples, the method 1600 may further comprise the receiving externally monitored patient information comprising receiving sensed physiologic information including at least one of: cardiac information; respiratory information; chest motion; oxygen desaturation information; peripheral arterial information; blood pressure; body position; and acoustic information including snoring.

In some examples, the method 1600 may further comprise the receiving the externally monitored patient information comprising receiving determined information including at least one of: sleep disorder breathing (SDB)-related index information; sleep information; and cardiac information.

As shown at 1604 in FIG. 22, in some examples method 1600 comprises determining or predicting one or more likely causes of current or prospective non-adherence based upon a review or comparison of the usage information with previous patient usage information or a model thereof. In some embodiments, the determined or predicted cause(s) of patient non-adherence can be displayed to a clinician. Alternatively or in addition, the determined or predicted likely causes of patient non-adherence and/or information utilized to arrive at the determined or predicted cause(s) of patient non-adherence can initiate other methods of the present disclosure.

FIG. 23 is a flow diagram schematically representing an example method 1700 of patient management. In some examples, method 1700 may be implemented via at least some the example arrangements, patient remote, patient app, processing resource, clinician portals, engines, parameters, functions, user interfaces, control portions, etc., as described in association with at least FIGS. 1-22. In some examples, method 1700 may be implemented via at least some elements, arrangements, patient remote, patient app, processing resource, clinician portals, engines, parameters, functions, user interfaces, control portions, etc. other than those described in association with at least FIGS. 1-22.

As shown at 1702 in FIG. 23, in some examples method 1700 comprises receiving, via a processing resource, usage information of a patient under review from a patient app or other resource. In some such examples, the method may comprise the patient app receiving or generating the usage information from a patient remote control. The patient remote control may receive at least some of the patient information from an implantable medical device and/or may produce some of the patient information from the patient remote control tracking and storing patient usage of the patient remote control.

In some examples, the method 1700 may further comprise the receiving externally monitored patient information comprising receiving sensed physiologic information including at least one of: cardiac information; respiratory information; chest motion; oxygen desaturation information; peripheral arterial information; blood pressure; body position; and acoustic information including snoring.

In some examples, the method 1700 may further comprise the receiving the externally monitored patient information comprising receiving determined information including at least one of: sleep disorder breathing (SDB)-related index information; sleep information; and cardiac information.

As shown at 1704 in FIG. 23, in some examples method 1700 comprises determining recommended action(s) to address current or potential patient non-adherence based upon a review or comparison of the usage information with previous patient usage information or a model thereof. In some embodiments, the determined recommended action(s) can be displayed to a clinician. Alternatively or in addition, the determined recommended actions information utilized to arrive at the determined recommended action(s) can initiate other methods of the present disclosure.

In some non-limiting examples above, reference is made to stimulating an IHN-innervating nerve and/or hypoglossal nerve. With this in mind, FIG. 24 is a diagram 9900 schematically representing patient anatomy and providing further details regarding example devices and/or example methods for stimulating an IHM-innervating nerve and/or hypoglossal nerve. As shown in FIG. 24, diagram 9900 includes a side view schematically representing an AC-main nerve 9915, in context with a hypoglossal nerve 9905 and with cranial nerves C1, C2, C3. As shown in FIG. 24, portion 9929A of the AC-main nerve 9915 (e.g. a portion or trunk connecting to the AC loop nerve 9919) extends anteriorly from a first cranial nerve C1 with a segment 9917 running alongside (e.g. coextensive with) the hypoglossal nerve 9905 for a length until the AC-main nerve 9915 diverges from the hypoglossal nerve 9905 to form a superior root 9925 of the AC-main nerve 9915, which forms part of the AC loop nerve 9919. A portion of the hypoglossal nerve 9905 extends distally to innervate the genioglossus muscle 9904. As further shown in FIG. 24, the superior root 9925 of the AC-main nerve 9915 extends inferiorly (i.e. downward) until reaching near bottom portion 9918 of the AC loop nerve 9919, from which the AC loop nerve 9919 extends superiorly (i.e. upward) to form an lesser root 9927 (i.e. inferior root) which joins to the second and third cranial nerves, C2 and C3, respectively and via portions 9929B, 9929C.

As further shown in FIG. 24, several branches 9931 extend off the AC loop nerve 9919, including branch 9932 which innervates the omohyoid muscle group 9934, branch 9942 which innervates the sternothyroid muscle group 9944 and at least a portion (e.g. inferior portion) of the sternohyoid muscle group 9954. Another branch 9952, near bottom portion 9918 of the AC loop nerve 9919, innervates at least a portion (e.g. superior portion) of the sternohyoid muscle group 9954. In some examples, the collective arrangement of the AC-main nerve 9915 (including at least superior root 9925 of the AC loop nerve 9919) and its related branches (e.g. at least 9932, 9942, 9952) when considered together, or any of those elements individually, may sometimes be referred to as an IHM-innervating nerve 9916. It will be further understood that at least one such IHM-innervating nerve 9916 is present on both sides (e.g. right and left) of the patient's body.

Stimulation may be delivered to many different locations of an IHM-innervating nerve 9916/nerve branches. Of these various potential stimulation locations, FIG. 24 generally illustrates three example stimulation locations A, B, and C. A stimulation element may be placed at all three of these locations or just some (e.g. one or two) of these example stimulation locations. At each location, a wide variety of types of stimulation elements (e.g. cuff electrode, axial array, paddle electrode, etc.) may be implanted depending on the particular delivery path, method, etc. For example, any one or a combination of the various example stimulation elements (and associated manner of access, delivery, etc.) described in association with at least FIG. 1 may be used to deliver such stimulation. In some such examples, a scale of the various stimulation elements, anchors, access tools, and/or other elements in some of the examples in FIG. 1 may be reduced to accommodate a generally smaller diameter of the IHM-innervating nerve/nerve branches 9916 as compared to some other nerve portions, such as at least some portions of the hypoglossal nerve.

With further reference to FIG. 24, at each example stimulation A, B, C, a stimulation element may be delivered subcutaneously, intravascularly, etc. At each stimulation location, in some examples the stimulation element may comprise a microstimulator.

It will be understood that these example stimulation locations A, B, C are not limiting and that other portions along the IHM-innervating nerve 9916/nerve branches may comprise suitable stimulation locations, depending on the particular objectives of the stimulation therapy, on the available access/delivery issues, etc.

Among the different physiologic effects resulting from stimulation of the various portions of the IHM-innervating nerve 9916/nerve branches (and/or innervated muscle portions, neuromuscular junctions, etc.), in some examples stimulation of nerve branches which cause contraction of the sternothyroid muscle and/or the sternohyoid muscle may cause the larynx to be pulled inferiorly, which in turn may increase and/or maintain upper airway patency in at least some patients. Such stimulation may be applied without stimulation of the hypoglossal nerve or may be applied in coordination with stimulation of the hypoglossal nerve 9905.

The following examples may comprise at least some of substantially the same features and attributes as, and/or example implementations of, the previously described examples of the present disclosure. The following examples may be implemented alone or together, which may comprise any various complementary combinations.

Example 1. A method comprising: automatically obtaining information relating to a patient's usage of an implantable medical device in delivering stimulation energy to the patient over a first period of time; and determining a likelihood of the patient to adhere to a treatment plan for use of the implantable medical device after the first period of time based upon the obtained information.

Example 2. The method of example 1, wherein the step of determining includes comparing the obtained information with a database of usage information obtained from a plurality of other patients.

Example 3. The method of example 2, wherein the step of determining includes matching the obtained information as input patterns into a machine learning model to generate an individual adherence prediction for the patient.

Example 4. The method of example 2, wherein the machine learning model is a pre-trained deep learning model.

Example 5. The method of example 3, wherein the machine learning model reviews relationships of usage patterns over period of time akin to the first period of time and long term adherence to a treatment plan akin to the treatment plan for at least five hundred other patients.

Example 6. The method of example 3, wherein the first period of time is less than three months following initial implant of the implantable medical device, and further wherein the step of determining predicts whether the patient will have high or low daily usage, on average, of the implantable medical device in delivering stimulation therapy more than three months after initial implant.

Example 7. The method of example 6, wherein high daily usage is designated as more than four hours, and low daily usage is designated as less than four hours.

Example 8. The method of example 6, wherein high daily usage is designated as more than five days per week, and low daily usage is designated as less than five days per week.

Example 9. The method of example 6, wherein high daily usage is designated as the combination of more than four hours on at least five days within a week, and low usage is designated as less four hours on fewer than five nights per week.

Example 10. The method of example 3, wherein the step of determining predicts wherein the first period of time is less than three months following initial implant of the implantable medical device, and further wherein the step of determining predicts whether the patient will have high or low daily usage, on average, of the implantable medical device in delivering stimulation therapy three months after initial implant.

Example 11. The method of example 10, wherein high daily usage is designated as more than four hours, and low daily usage is designated as less than four hours.

Example 12. The method of example 10, wherein high daily usage is designated as more than five days per week, and low daily usage is designated as less than five days per week.

Example 13. The method of example 10, wherein high daily usage is designated as the combination of more than four hours on at least five days within a week, and low usage is designated as less four hours on fewer than five nights per week.

Example 14. The method of example 1, wherein operation of the implantable medical device is at least partially controllable by the patient via a patient remote following implant of the implantable medical device.

Example 15. The method of example 14, wherein the obtained information includes timestamped information from the patient remote indicative of patient-prompted operation of the implantable medical device.

Example 16. The method of example 15, wherein the patient remote is operable by the patient to prompt delivery of a “therapy on” command to the implantable medical device that initiates performance of a therapy session in which the implantable medical device operates, in accordance with instructions programed to the implantable medical device, to continuously deliver pulsed stimulation energy to the patient, and further wherein the timestamped information includes timestamped therapy on commands during the first period of time.

Example 17. The method of example 16, wherein the patient remote is operable by the patient to prompt delivery of a “therapy off” command to the implantable medical device that terminates performance of a previously-initiated therapy session, and further wherein the timestamped information includes timestamped therapy off commands during the first period of time.

Example 18. The method of example 17, wherein the timestamped information is indicative of a duration of each therapy session during the first period of time.

Example 19. The method of example 17, wherein the patient remote is operable by the patient to prompt delivery of a “pause” command to the implantable medical device that temporarily pauses delivery of stimulation energy to the patient as part of a previously-initiated therapy session for a pre-determined period of time, and further wherein the timestamped information includes timestamped therapy pause commands during the first period of time.

Example 20. The method of example 19, wherein the patient remote is operable by the patient to prompt delivery of an “intensity change” instruction to the implantable medical device that can change an intensity of stimulation energy delivered during a particular therapy session, and further wherein the timestamped information includes intensity change commands during the first period of time.

Example 21. The method of example 20, wherein the timestamped information includes a maximum amplitude of stimulation energy delivered during each individual therapy session during the first period of time.

Example 22. The method of example 1, wherein following initial implant and programming of the implantable medical device, the patient begins normal use of the implantable medical device on a first day, and further wherein the first period of time is not greater than at least one of a therapy maintenance phase or a therapy adjustment phase.

Example 23. The method of example 22, wherein the step of determining includes estimating the likelihood of the patient to adhere to the treatment plan at a point in time more than 90 days from the first day.

Example 24. The method of example 23, wherein the step of determining includes estimating the likelihood of the patient to adhere to the treatment plan at a point in time less one year from the first day.

Example 25. The method of example 23, wherein the step of determining includes estimating the likelihood of the patient to adhere to the treatment plan at a point in time less than 90 days from the first day.

Example 26. The method of example 1, wherein the likelihood of the patient adhering to the treatment plan is based, at least in part, upon the number of days the patient did not use the implantable medical device over the first period of time.

Example 27. The method of example 1, wherein the implantable medical device is operable to continuously deliver pulsed stimulation energy to the patient over the course of a therapy session, and further wherein the likelihood of the patient to adhere to the treatment plan is based, at least in part, upon the duration of each therapy session occurring over the first period of time.

Example 28. The method of example 27, wherein the likelihood of the patient to adhere to the treatment plan is based, at least in part, upon the number of therapy sessions with a duration of more than 4 hours and the number of therapy sessions with a duration of less than 4 hours.

Example 29. The method of example 27, wherein the likelihood of the patient to adhere to the treatment plan is based, at least in part, upon the number of therapy sessions in at least 5 of the past 7 days, and a number of therapy sessions less than 5 of the past 7 days.

Example 30. The method of example 27, wherein the likelihood of the patient to adhere to the treatment plan is based, at least in part, upon the number of therapy sessions in at least 5 of the past 7 days and with a duration of more than 4 hours, and a number of therapy sessions less than 5 of the past 7 days with a duration of less than 4 hours.

Example 31. The method of example 27, wherein the implantable medical device is operable to temporarily pause delivery of stimulation energy to the patient for a pre-determined period of time during a particular therapy session, and further wherein the likelihood of the patient to adhere to the treatment plan is based, at least in part, upon the number of pauses in each of the therapy sessions or total pause time over the first period of time.

Example 32. The method of example 27, wherein the likelihood of the patient to adhere to the treatment plan is based, at least in part, upon an intensity of stimulation energy delivered during each of the therapy sessions.

3 Example 3. The method of example 32, wherein the likelihood of the patient to adhere to the treatment plan is based, at least in part, upon a maximum amplitude of stimulation energy delivered during each of the therapy sessions.

Example 34. The method of example 32, wherein the likelihood of the patient to adhere to the treatment plan is based, at least in part, upon changes in stimulation intensity as selected by the patient.

Example 35. The method of example 1, wherein the likelihood of the patient adhering to the treatment plan is based, at least in part, on therapy start and stop times, and derived metrics such as total therapy hours of when the patient used the implantable medical device over the first period of time.

Example 36. The method of example 1, further comprising: identifying one or more potential causes of a determined low likelihood of the patient to adhere to a treatment plan.

Example 37. The method of example 36, wherein the one or more potential causes is selected from the group consisting of patient sleep issue, stimulation-caused patient discomfort, proper sleep remote operation, and efficacy of treatment.

Example 38. The method of example 37, wherein the patient sleep issue is selected from the group consisting of insomnia, circadian sleep disorders, irregular timing of sleep, and irregular length of sleep.

Example 39. The method of example 36, wherein the step of identifying includes applying at least one of a group analysis, cohort analysis, and a cluster analysis to the obtained information.

Example 40. The method of example 39, wherein the analysis includes finding similar patterns in the determined daily usage of the implantable medical device, length of time of usage of the implantable medical device, and consistency of start time of usage of the implantable medical device by the patient over the first period of time.

Example 41. The method of example 36, wherein the step of identifying includes identifying two or more potential causes, the method further comprising automatically prioritizing the identified two or more causes.

Example 42. The method of example 41, wherein the step of identifying further includes determining a standard deviation in two or more usage-related parameters implicated by the obtained information over the first period of time, and designating the usage-related parameter with a greatest standard deviation as having priority over other usage-related parameters.

Example 43. The method of example 42, wherein the usage-related parameters are selected from the group consisting of patient-prompted therapy off, therapy sessions of less than one hour, number of therapy pause prompts, number of missed therapy sessions, length of therapy sessions, total therapy off time, total therapy on time, and sleep.

Example 44. The method of example 36, wherein the implantable medical device is operable in a therapy on state which stimulation energy is delivered to the patient and a therapy off state in which stimulation energy is not delivered to the patient, the therapy on state including an optional therapy pause mode in which delivery of stimulation energy is temporarily paused, and further where a patient usage session begins when the implantable medical device is transitioned from the therapy off state to the therapy on state and ends when the implantable medical device is subsequently transitioned to the therapy off state, and further wherein the step of identifying includes: determining a sleep apnea severity value for at least some of the patient usage sessions occurring over the first period of time; recording a maximum stimulation amplitude for at least some of the patient usage sessions occurring over the first period of time; and correlating the determined sleep apnea severity value with the maximum stimulation amplitude for the corresponding patient usage sessions.

Example 45. The method of example 44, wherein the sleep apnea severity value is an Apnea Hypopnea Index (AHI).

Example 46. The method of example 44, wherein the sleep apnea severity value is an Oxygen Desaturation Index (ODI).

Example 47. The method of example 44, wherein the sleep apnea severity value is minutes of snoring.

Example 48. The method of example 44, wherein the sleep apnea severity value is number snoring occurrences per hour.

Example 49. The method of example 44, wherein the sleep apnea severity value is a patient-reported measurement of sleepiness, such as the Epworth Sleepiness Scale.

Example 50. The method of example 44, wherein the sleep apnea severity value is a patient-reported measurement of daytime activity, such as the Functional Outcomes of Sleep Questionnaire.

Example 51. The method of example 44, wherein the step of identifying further includes designating a negative correlation under circumstances where an increase in maximum stimulation amplitude does not provide a corresponding improvement in the sleep apnea severity value.

Example 52. The method of example 44, wherein the step of correlating includes determining a correlation coefficient to measure a linear relationship between the determined sleep apnea severity value and the recorded maximum stimulation amplitude over time.

Example 53. The method of example 52, wherein the step of identifying further includes designating the recorded maximum stimulation amplitude corresponding with a change in the correlation coefficient from positive to negative as a possible amplitude discomfort issue.

Example 54. The method of example 52, wherein the step of correlating includes plotting the determined correlation coefficient over time, and further wherein the step of identifying further includes designating the recorded maximum stimulation amplitude corresponding with a slope of the plotted correlation coefficient becoming increasingly negative.

Example 55. The method of example 36, wherein the implantable medical device is operable in a therapy on state which stimulation energy is delivered to at least one stimulation site of the patient and a therapy off state in which stimulation energy is not delivered to the at least one stimulation site, the therapy on state including an optional therapy pause mode in which delivery of stimulation energy is temporarily paused, and further where a patient usage session begins when the implantable medical device is transitioned from the therapy off state to the therapy on state and ends when the implantable medical device is subsequently transitioned to the therapy off state, and further wherein the step of identifying includes: recording a length of session time of each of the patient usage sessions occurring over the first period of time; recording a maximum stimulation amplitude of each of the patient usage sessions occurring over the first period of time; and correlating the length of session time the maximum stimulation amplitude for the corresponding patient usage sessions.

Example 56. The method of example 55, wherein the step of identifying further includes designating a negative correlation under circumstances where an increase in maximum stimulation amplitude does corresponds with a decrease in the corresponding length of session time.

Example 57. The method of example 56, wherein the step of correlating includes determining a correlation coefficient to measure a linear relationship between the recorded length of session time and the recorded maximum stimulation amplitude over time.

Example 58. The method of example 57, wherein the step of identifying further includes designating the recorded maximum stimulation amplitude corresponding with a change in the correlation coefficient from positive to negative as a possible amplitude discomfort issue.

Example 59. The method of example 56, wherein the step of correlating includes plotting the determined correlation coefficient over time, and further wherein the step of identifying further includes designating the recorded maximum stimulation amplitude corresponding with a slope of the plotted correlation coefficient becoming increasingly negative.

Example 60. The method of example 59, wherein the step of correlating further includes: determining a plurality of aggregated correlations, each aggregated correlation representing a correlation of a series of consecutive therapy usage sessions based upon a normalized maximum stimulation amplitude and a normalized length of session time for each of the therapy usage sessions of the series of consecutive usage sessions; and determining, for each of the therapy usage sessions, whether the patient is in a discomfort zone or a relief zone based upon the corresponding correlation coefficient, aggregated correlation and slope.

Example 61. The method of example 36, further comprising: informing a clinician of the determined likelihood of the patient to adhere to the treatment plan and the identified one or more potential causes of a determined low likelihood of the patient to adhere to the treatment plan via a display of a clinician device.

Example 62. The method of example 36, further comprising: automatically generating one or more recommended actions to address the identified one or more potential causes.

Example 63. The method of example 62, wherein the step of automatically generating includes selecting a recommended action for an identified potential cause from the group consisting of: one or more of sleep hygiene education, sleep reminders, and sleep goal setting for an identified potential cause of poor sleep habits; one or more of cognitive behavior therapy and pharmaceuticals for an identified potential cause of insomnia; and one or more of amplitude goal setting, stimulation re-programming, implementing limits on patient-prompted stimulation amplitude increases, temporarily preventing patient-prompted stimulation amplitude increases, and cognitive behavior therapy for an identified potential cause of stimulation-related discomfort.

Example 64. The method of example 62, further comprising: displaying information relating to the generated recommended action on a patient device.

Example 65. The method of example 1, wherein the implantable medical device is one of a fully implantable medical device and an implantable medical device with at least one component external the patient

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method comprising:
automatically obtaining information relating to a patient's usage of an implantable medical device in delivering stimulation energy to the patient over a first period of time; and
determining a likelihood of the patient to adhere to a treatment plan for use of the implantable medical device after the first period of time based upon the obtained information.

2. The method of claim 1, wherein the implantable medical device is one of a fully implantable medical device and an implantable medical device with at least one component external the patient.

3. The method of claim 1, wherein the step of determining includes comparing the obtained information with a database of usage information obtained from a plurality of other patients.

4. The method of claim 3, wherein the step of determining includes matching the obtained information as input patterns into a machine learning model to generate an individual adherence prediction for the patient.

5. The method of claim 4, wherein the machine learning model reviews relationships of usage patterns over period of time akin to the first period of time and long term adherence to a treatment plan akin to the treatment plan for at least five hundred other patients.

6. The method of claim 4, wherein the first period of time is less than three months following initial implant of the implantable medical device, and further wherein the step of determining predicts whether the patient will have high or low daily usage, on average, of the implantable medical device in delivering stimulation therapy three months after initial implant.

7. The method of claim 1, wherein the implantable medical device is operable to continuously deliver pulsed stimulation energy to the patient over the course of a therapy session, and further wherein the likelihood of the patient to adhere to the treatment plan is based, at least in part, upon the duration of each therapy session occurring over the first period of time.

8. The method of claim 7, wherein the implantable medical device is operable to temporarily pause delivery of stimulation energy to the patient for a pre-determined period of time during a particular therapy session, and further wherein the likelihood of the patient to adhere to the treatment plan is based, at least in part, upon the number of pauses in each of the therapy sessions or total pause time over the first period of time.

9. The method of claim 7, wherein the likelihood of the patient to adhere to the treatment plan is based, at least in part, upon an intensity of stimulation energy delivered during each of the therapy sessions.

10. The method of claim 7, wherein the likelihood of the patient to adhere to the treatment plan is based, at least in part, upon a maximum amplitude of stimulation energy delivered during each of the therapy sessions.

11. The method of claim 7, wherein the likelihood of the patient to adhere to the treatment plan is based, at least in part, upon changes in stimulation intensity as selected by the patient.

12. The method of claim 1, wherein the likelihood of the patient adhering to the treatment plan is based, at least in part, on therapy start and stop times, and derived metrics such as total therapy hours of when the patient used the implantable medical device over the first period of time.

13. The method of claim 1, further comprising:
identifying one or more potential causes of a determined low likelihood of the patient to adhere to a treatment plan.

14. The method of claim 13, wherein the one or more potential causes is selected from the group consisting of patient sleep issue, stimulation-caused patient discomfort, proper sleep remote operation, and efficacy of treatment.

15. The method of claim 14, wherein the patient sleep issue is selected from the group consisting of insomnia, circadian sleep disorders, irregular timing of sleep, and irregular length of sleep.

16. The method of claim 13, wherein the step of identifying includes applying at least one of a group analysis, cohort analysis, and a cluster analysis to the obtained information.

17. The method of claim 13, wherein the step of identifying includes identifying two or more potential causes, the method further comprising automatically prioritizing the identified two or more causes.

18. The method of claim 13, wherein the implantable medical device is operable in a therapy on state which stimulation energy is delivered to at least one stimulation site of the patient and a therapy off state in which stimulation energy is not delivered to the at least one stimulation site, the therapy on state including an optional therapy pause mode in which delivery of stimulation energy is temporarily paused, and further where a patient usage session begins when the implantable medical device is transitioned from the therapy off state to the therapy on state and ends when the implantable medical device is subsequently transitioned to the therapy off state, and further wherein the step of identifying includes:
determining a sleep apnea severity value for at least some of the patient usage sessions occurring over the first period of time;
recording a maximum stimulation amplitude for at least some of the patient usage sessions occurring over the first period of time; and
correlating the determined sleep apnea severity value with the maximum stimulation amplitude for the corresponding patient usage sessions.

19. The method of claim 18, wherein the sleep apnea severity value is at least one of:
an Apnea Hypopnea Index (AHI);
an Oxygen Desaturation Index (ODI);
minutes of snoring;
number of snoring occurrences per hour;
a patient-reported measurement of sleepiness; and
a patient-reported measurement of daytime activity.

20. The method of claim 18, wherein the step of correlating includes determining a correlation coefficient to measure a linear relationship between the determined sleep apnea severity value and the recorded maximum stimulation amplitude over time.
